(19) United States
(12) Patent Application Publication
(10) Pub. No.: US 2023/0000675 A1
(43) Pub. Date: Jan. 5, 2023

(54) DEVICE FOR ADMINISTERING DRUG TO EYELID

(71) Applicant: Azura Ophthalmics Ltd., Tel Aviv (IL)

(72) Inventors: Yair ALSTER, Tel Aviv (IL); Omer RAFAELI, Udim (IL); Marc GLEESON, Longueville (AU); Charles BOSWORTH, Las Vegas, NV (US); Eitan SHARIFF, Tel Aviv (IL); Dan TEBOUL, Tel Aviv (IL)

(21) Appl. No.: 17/858,729

(22) Filed: Jul. 6, 2022

Related U.S. Application Data
(63) Continuation of application No. PCT/IB2021/000007, filed on Jan. 7, 2021.
(60) Provisional application No. 62/958,605, filed on Jan. 8, 2020.
(60) Provisional application No. 62/959,717, filed on Jan. 10, 2020.

(51) Int. Cl. A61F 9/00; A61K 9/00

(52) U.S. Cl. CPC A61F 9/0017; A61K 9/0051; A61F 2250/0067

(57) ABSTRACT

Provided herein are devices and methods for applying a medicament to an eyelid, e.g. eyelid margin, of a patient while protecting the adjunct ocular tissues such as the cornea.

### CROSS-REFERENCE TO RELATED APPLICATIONS

This application is a continuation of International Application No. PCT/IB2021/000007, filed on Jan. 7, 2021, which claims the benefit of U.S. Provisional Application No. 62/958,605, filed Jan. 8, 2020 and U.S. Provisional Application No. 62/959,717, filed Jan. 10, 2020, all of which are incorporated herein by reference in their entireties.

### BACKGROUND

The margin of the eyelid is the area of the anterior eye around the palpebral fissure where the outer oily-dry skin on the outside of the eyelids and body transforms into the aqueous-moist mucosa of the conjunctiva at the ocular surface. Meibomian oil glands, which are located inside the eyelid, deliver their oil onto the Fralid margin.

Many ocular diseases that are symptomatic in the eye can be treated or modulated administering a medicament to the eyelid. Meibomian gland dysfunction (MGD) can be caused by keratinized material blocking the orifices of the meibomian glands. Blepharitis eyelid disorders are characterized by erythematous eyelids with accumulation of debris along the eyelid margin. Malignant eyelid tumors may be associated with lash loss and erosion of normal eyelid structures. Other eyelid disorders include blepharospasm, styes, chalazion, meibomianitis, lacrimal duct obstruction, seborrheic keratosis, actinic keratosis, hidrocystoma, molluscum contagiosum, nevus, xanthelasma, blepharoptosis, coloboma, dermatochalasis, ectropion, entropion, facial palsy, and trichiasis. In some embodiments, administration is to the eyelid and not the ocular surface. In some embodiments, the medicament is applied to the eyelid and not the ocular surface. In some embodiments the ocular surface is protected by a device (e.g. a shield) as described herein. In some embodiments there is a desire to allow the medicament to stay on the eyelid (e.g., eyelid margin) and not move from that location

### SUMMARY

Provided herein are methods and devices for administering a medicament to the eyelid (e.g., upper eyelid, lower eyelid, eyelid margin, or any combination thereof) of an individual, such as to treat an ocular disorder. In some instances, the medicament is administered (or the device is configured to provide the medicament) to the eyelid without subjecting or exposing the ocular surface to the medicament. In certain embodiments, the device comprises a first portion comprising a handle and a second portion comprising a sealing element. In certain embodiments, the second portion consists of a sealing element. In certain embodiments, the first portion comprises a medicament surface. The medicament surface can comprise a medicament recession. In certain embodiments, the ocular disorder is a disorder that demonstrates ocular symptoms (e.g., dry eye and/or ocular irritation). In specific embodiments, the ocular disorder is a disorder mediated by a dermal or glandular mechanism or disorder (e.g., meibomian gland dysfunction (MGD)). In some instances, treatment of ocular disorders mediated by dermal, glandular, or other lid disorders by administering a medicament to the eyelid, but not the ocular surface, allows for the administration of medicaments that may not otherwise be ophthalmically acceptable or desirable. In certain instances, a method provided herein utilized and/or the device provided herein is any suitable device, such as any one of the devices described herein. In certain instances, medicaments tend to move around over the ocular area (e.g. fornix or cornea) and there is a desire to enable the medicament to remain on the lid to achieve maximal therapeutic effect.

In one aspect, disclosed herein is a device for applying a medicament to an eyelid (e.g., of an individual in need thereof, such as an individual suffering from an ocular or lid disorder) comprising (or a method of administering a medicament to an eyelid with a device comprising): a first portion having a distal end and a proximal end; wherein the first portion comprises the medicament surface close to the distal end, wherein the first portion is shaped and dimensioned to allow close contact between an eye lid margin and the medicament surface for an extended period of time without any additional support provided on the first portion proximal to the medicament surface.

Another aspect provided herein is a device for applying a medicament to an eyelid (e.g., of an individual in need thereof, such as an individual suffering from an ocular or lid disorder) comprising (or a method of administering a medicament to an eyelid with a device comprising): a first portion having a distal end and a proximal end; a second portion configured to be inserted beneath an eyelid of a patient; and the medicament surface on the first portion and at an intersection of the first portion and the second portion, wherein the medicament surface contacts at least a section of the eyelid of the patient when the second portion is inserted beneath the eyelid of the patient.

Further, provided herein is a device for applying a medicament to an eyelid comprising a medicament surface configured to absorb or contain the medicament, wherein the medicament surface contacts at least a section of the eyelid of the patient when the device is inserted between the eyelids of a patient.

In some embodiments, the section of the eyelid of the patient is the lid margin of the eyelid of the patient. In some embodiments, the first portion is generally flat. In some embodiments, the first portion comprises a grip protrusion. In some embodiments, the second portion comprises a distal surface configured to contact an eye of the patient, and a proximal surface configured for contact with an inner eyelid of the patient. In some embodiments, the distal surface has a roughness of less than about 2.5 nm. In some embodiments, the distal surface conforms to the eye of the patient. In some embodiments, the distal surface is concave.

In some embodiments, the distal surface has a radius of curvature of about 7 mm to about 18 mm. In some embodiments, the distal surface has a radius of curvature of about 7 mm to about 8 mm, about 7 mm to about 9 mm, about 7 mm to about 10 mm, about 7 mm to about 12 mm, about 7 mm to about 14 mm, about 7 mm to about 16 mm, about 7 mm to about 18 mm, about 8 mm to about 9 mm, about 8 mm to about 10 mm, about 8 mm to about 12 mm, about 8 mm to about 14 mm, about 8 mm to about 16 mm, about 8 mm to about 18 mm, about 9 mm to about 10 mm, about 9 mm to about 12 mm, about 9 mm to about 14 mm, about 9 mm to about 16 mm, about 9 mm to about 18 mm, about 10 mm to about 12 mm, about 10 mm to about 14 mm, about 10 mm to about 16 mm, about 10 mm to about 18 mm, about 12 mm to about 14 mm, about 12 mm to about 16 mm, about 12 mm to about 18 mm, about 14 mm to about 16 mm, about 14 mm to about 18 mm, or about 16 mm to about 18 mm. In some embodiments, the distal surface has a radius of curvature of about 7 mm, about 8 mm, about 9 mm, about 10 mm, about 12 mm, about 14 mm, about 16 mm, or about 18 mm. In some embodiments, the distal surface has a radius of curvature of at least about 7 mm, about 8 mm, about 9 mm, about 10 mm, about 12 mm, about 14 mm, or about 16 mm. In some embodiments, the distal surface has a radius of curvature of at most about 8 mm, about 9 mm, about 10 mm, about 12 mm, about 14 mm, about 16 mm, or about 18 mm.

In some embodiments, the second portion is further configured to prevent the medicament deposited on the medicament surface from contacting the eye of the patient. In some embodiments, at least part of the second portion is covered by an absorbing material. In some embodiments, the absorbing material is configured to absorb the medicament. In some embodiments, the absorbing material is configured to prevent the medicament deposited on the medicament surface from contacting the eye of the patient. In some embodiments, the second portion comprises a sealing protrusion, a sealing recess, or both. In some embodiments, at least one of the sealing protrusions and the sealing recess is configured to prevent the medicament deposited on the medicament surface from contacting the eye of the patient.

In some embodiments, a ratio between a thickness of the second portion protrusion and a height of the sealing protrusion or a depth of the sealing recess is about 1.5:1 to about 10:1. In some embodiments, a ratio between a thickness of the second portion protrusion and a height of the sealing protrusion or a depth of the sealing recess is about 1.5:1 to about 2:1, about 1.5:1 to about 3:1, about 1.5:1 to about 4:1, about 1.5:1 to about 5:1, about 1.5:1 to about 6:1, about 1.5:1 to about 7:1, about 1.5:1 to about 8:1, about 1.5:1 to about 9:1, about 1.5:1 to about 10:1, about 2:1 to about 3:1, about 2:1 to about 4:1, about 2:1 to about 5:1, about 2:1 to about 6:1, about 2:1 to about 7:1, about 2:1 to about 8:1, about 2:1 to about 9:1, about 2:1 to about 10:1, about 3:1 to about 4:1, about 3:1 to about 5:1, about 3:1 to about 6:1, about 3:1 to about 7:1, about 3:1 to about 8:1, about 3:1 to about 9:1, about 3:1 to about 10:1, about 4:1 to about 5:1, about 4:1 to about 6:1, about 4:1 to about 7:1, about 4:1 to about 8:1, about 4:1 to about 9:1, about 4:1 to about 10:1, about 5:1 to about 6:1, about 5:1 to about 7:1, about 5:1 to about 8:1, about 5:1 to about 9:1, about 5:1 to about 10:1, about 6:1 to about 7:1, about 6:1 to about 8:1, about 6:1 to about 9:1, about 6:1 to about 10:1, about 7:1 to about 8:1, about 7:1 to about 9:1, about 7:1 to about 10:1, about 8:1 to about 9:1, about 8:1 to about 10:1, or about 9:1 to about 10:1. In some embodiments, a ratio between a thickness of the second portion protrusion and a height of the sealing protrusion or a depth of the sealing recess is about 1.5:1, about 2:1, about 3:1, about 4:1, about 5:1, about 6:1, about 7:1, about 8:1, about 9:1, or about 10:1. In some embodiments, a ratio between a thickness of the second portion protrusion and a height of the sealing protrusion or a depth of the sealing recess is at least about 1.5:1, about 2:1, about 3:1, about 4:1, about 5:1, about 6:1, about 7:1, about 8:1, or about 9:1. In some embodiments, a ratio between a thickness of the second portion protrusion and a height of the sealing protrusion or a depth of the sealing recess is at most about 2:1, about 3:1, about 4:1, about 5:1, about 6:1, about 7:1, about 8:1, about 9:1, or about 10:1.

In some embodiments, the second portion comprises a primary second portion and a secondary second portion. In some embodiments, the first portion intersects the second portion at the intersection of the primary second portion and the secondary second portion. In some embodiments, the primary second portion is configured to be inserted beneath an upper eyelid of the patient and wherein the secondary second portion is configured to be inserted beneath a lower eyelid of the patient. In some embodiments, the second portion is configured to be inserted beneath an upper eyelid of the patient, a lower eyelid of the patient, or both. In some embodiments, the medicament surface comprises a primary medicament surface at an intersection of the first portion and the primary second portion and a secondary medicament surface at an intersection of the first portion and the secondary second portion. In some embodiments, the primary medicament surface contacts at least a portion of an upper eyelid of the patient. In some embodiments, the secondary medicament surface contacts at least a portion of a lower eyelid of the patient when the second portion is inserted beneath the eyelid of the patient. In some embodiments, an outer edge of the second portion is rounded. In some embodiments, the first portion and the second portion are generally coplanar. In some embodiments, the first portion and the second portion are generally perpendicular.

In some embodiments, an angle between the first portion and the second portion at the intersection of the first portion and the second portion is about 90 degrees to about 180 degrees. In some embodiments, an angle between the first portion and the second portion at the intersection of the first portion and the second portion is about 90 degrees to about 100 degrees, about 90 degrees to about 120 degrees, about 90 degrees to about 130 degrees, about 90 degrees to about 140 degrees, about 90 degrees to about 150 degrees, about 90 degrees to about 160 degrees, about 90 degrees to about 170 degrees, about 90 degrees to about 180 degrees, about 100 degrees to about 120 degrees, about 100 degrees to about 130 degrees, about 100 degrees to about 140 degrees, about 100 degrees to about 150 degrees, about 100 degrees to about 160 degrees, about 100 degrees to about 170 degrees, about 100 degrees to about 180 degrees, about 120 degrees to about 130 degrees, about 120 degrees to about 140 degrees, about 120 degrees to about 150 degrees, about 120 degrees to about 160 degrees, about 120 degrees to about 170 degrees, about 120 degrees to about 180 degrees, about 130 degrees to about 140 degrees, about 130 degrees to about 150 degrees, about 130 degrees to about 160 degrees, about 130 degrees to about 170 degrees, about 130 degrees to about 180 degrees, about 140 degrees to about 150 degrees, about 140 degrees to about 160 degrees, about 140 degrees to about 170 degrees, about 140 degrees to about 180 degrees, about 150 degrees to about 160 degrees, about 150 degrees to about 170 degrees, about 150 degrees to about 180 degrees, about 160 degrees to about 170 degrees, about 160 degrees to about 180 degrees, or about 170 degrees to about 180 degrees. In some embodiments, an angle between the first portion and the second portion at the intersection of the first portion and the second portion is about 90 degrees, about 100 degrees, about 120 degrees, about 130 degrees, about 140 degrees, about 150 degrees, about 160 degrees, about 170 degrees, or about 180 degrees. In some embodiments, an angle between the first portion and the second portion at the intersection of the first portion and the second portion is at least about 90 degrees, about 100 degrees, about 120 degrees, about 130 degrees, about 140 degrees, about 150 degrees, about 160 degrees, or about 170 degrees. In some embodiments, an angle between the first portion and the second portion at the intersection of the first portion and the second portion is at most about 100 degrees, about 120 degrees, about 130 degrees, about 140 degrees, about 150 degrees, about 160 degrees, about 170 degrees, or about 180 degrees.

In some embodiments, the medicament surface is coplanar with at least one of the first portion and the second portion. In some embodiments, at least a portion of the medicament surface is perpendicular to at least one of the first portion and the second portion. In some embodiments, the medicament surface is concave. In some embodiments, the medicament surface comprises the medicament. In some embodiments, a thickness of the first portion is greater than or equal to a thickness of the second portion. In some embodiments, a thickness of the first portion is less than or equal to a thickness of the second portion. In some embodiments, a width of the first portion is greater than or equal to a thickness of the second portion. In some embodiments, a width of the first portion is less than or equal to a thickness of the second portion.

In some embodiments, a width of at least one of the first portion and the second portion is about 18 mm to about 36 mm. In some embodiments, a width of at least one of the first portion and the second portion is about 18 mm to about 20 mm, about 18 mm to about 22 mm, about 18 mm to about 24 mm, about 18 mm to about 26 mm, about 18 mm to about 28 mm, about 18 mm to about 30 mm, about 18 mm to about 32 mm, about 18 mm to about 34 mm, about 18 mm to about 36 mm, about 20 mm to about 22 mm, about 20 mm to about 24 mm, about 20 mm to about 26 mm, about 20 mm to about 28 mm, about 20 mm to about 30 mm, about 20 mm to about 32 mm, about 20 mm to about 34 mm, about 20 mm to about 36 mm, about 22 mm to about 24 mm, about 22 mm to about 26 mm, about 22 mm to about 28 mm, about 22 mm to about 30 mm, about 22 mm to about 32 mm, about 22 mm to about 34 mm, about 22 mm to about 36 mm, about 24 mm to about 26 mm, about 24 mm to about 28 mm, about 24 mm to about 30 mm, about 24 mm to about 32 mm, about 24 mm to about 34 mm, about 24 mm to about 36 mm, about 26 mm to about 28 mm, about 26 mm to about 30 mm, about 26 mm to about 32 mm, about 26 mm to about 34 mm, about 26 mm to about 36 mm, about 28 mm to about 30 mm, about 28 mm to about 32 mm, about 28 mm to about 34 mm, about 28 mm to about 36 mm, about 30 mm to about 32 mm, about 30 mm to about 34 mm, about 30 mm to about 36 mm, about 32 mm to about 34 mm, about 32 mm to about 36 mm, or about 34 mm to about 36 mm. In some embodiments, a width of at least one of the first portion and the second portion is about 18 mm, about 20 mm, about 22 mm, about 24 mm, about 26 mm, about 28 mm, about 30 mm, about 32 mm, about 34 mm, or about 36 mm. In some embodiments, a width of at least one of the first portion and the second portion is at least about 18 mm, about 20 mm, about 22 mm, about 24 mm, about 26 mm, about 28 mm, about 30 mm, about 32 mm, or about 34 mm. In some embodiments, a width of at least one of the first portion and the second portion is at most about 20 mm, about 22 mm, about 24 mm, about 26 mm, about 28 mm, about 30 mm, about 32 mm, about 34 mm, or about 36 mm.

In some embodiments, the second portion has a height of at least about 4 mm.

In some embodiments, at least one of the first portion and the second portion has a Young's modulus of about 0.05 MPa to about 10 MPa. In some embodiments, at least one of the first portion and the second portion has a Young's modulus of about 0.05 MPa to about 0.1 MPa, about 0.05 MPa to about 0.5 MPa, about 0.05 MPa to about 1 MPa, about 0.05 MPa to about 2 MPa, about 0.05 MPa to about 3 MPa, about 0.05 MPa to about 4 MPa, about 0.05 MPa to about 5 MPa, about 0.05 MPa to about 6 MPa, about 0.05 MPa to about 8 MPa, about 0.05 MPa to about 10 MPa, about 0.1 MPa to about 0.5 MPa, about 0.1 MPa to about 1 MPa, about 0.1 MPa to about 2 MPa, about 0.1 MPa to about 3 MPa, about 0.1 MPa to about 4 MPa, about 0.1 MPa to about 5 MPa, about 0.1 MPa to about 6 MPa, about 0.1 MPa to about 8 MPa, about 0.1 MPa to about 10 MPa, about 0.5 MPa to about 1 MPa, about 0.5 MPa to about 2 MPa, about 0.5 MPa to about 3 MPa, about 0.5 MPa to about 4 MPa, about 0.5 MPa to about 5 MPa, about 0.5 MPa to about 6 MPa, about 0.5 MPa to about 8 MPa, about 0.5 MPa to about 10 MPa, about 1 MPa to about 2 MPa, about 1 MPa to about 3 MPa, about 1 MPa to about 4 MPa, about 1 MPa to about 5 MPa, about 1 MPa to about 6 MPa, about 1 MPa to about 8 MPa, about 1 MPa to about 10 MPa, about 2 MPa to about 3 MPa, about 2 MPa to about 4 MPa, about 2 MPa to about 5 MPa, about 2 MPa to about 6 MPa, about 2 MPa to about 8 MPa, about 2 MPa to about 10 MPa, about 3 MPa to about 4 MPa, about 3 MPa to about 5 MPa, about 3 MPa to about 6 MPa, about 3 MPa to about 8 MPa, about 3 MPa to about 10 MPa, about 4 MPa to about 5 MPa, about 4 MPa to about 6 MPa, about 4 MPa to about 8 MPa, about 4 MPa to about 10 MPa, about 5 MPa to about 6 MPa, about 5 MPa to about 8 MPa, about 5 MPa to about 10 MPa, about 6 MPa to about 8 MPa, about 6 MPa to about 10 MPa, or about 8 MPa to about 10 MPa. In some embodiments, at least one of the first portion and the second portion has a Young's modulus of about 0.05 MPa, about 0.1 MPa, about 0.5 MPa, about 1 MPa, about 2 MPa, about 3 MPa, about 4 MPa, about 5 MPa, about 6 MPa, about 8 MPa, or about 10 MPa. In some embodiments, at least one of the first portion and the second portion has a Young's modulus of at least about 0.05 MPa, about 0.1 MPa, about 0.5 MPa, about 1 MPa, about 2 MPa, about 3 MPa, about 4 MPa, about 5 MPa, about 6 MPa, or about 8 MPa. In some embodiments, at least one of the first portion and the second portion has a Young's modulus of at most about 0.1 MPa, about 0.5 MPa, about 1 MPa, about 2 MPa, about 3 MPa, about 4 MPa, about 5 MPa, about 6 MPa, about 8 MPa, or about 10 MPa.

In some embodiments, at least one of the first portion and the second portion have a hardness of about 20 shore A to about 80 shore A. In some embodiments, at least one of the first portion and the second portion have a hardness of about 20 shore A to about 25 shore A, about 20 shore A to about 30 shore A, about 20 shore A to about 35 shore A, about 20 shore A to about 40 shore A, about 20 shore A to about 45 shore A, about 20 shore A to about 50 shore A, about 20 shore A to about 60 shore A, about 20 shore A to about 70 shore A, about 20 shore A to about 80 shore A, about 25 shore A to about 30 shore A, about 25 shore A to about 35 shore A, about 25 shore A to about 40 shore A, about 25 shore A to about 45 shore A, about 25 shore A to about 50 shore A, about 25 shore A to about 60 shore A, about 25 shore A to about 70 shore A, about 25 shore A to about 80 shore A, about 30 shore A to about 35 shore A, about 30 shore A to about 40 shore A, about 30 shore A to about 45 shore A, about 30 shore A to about 50 shore A, about 30 shore A to about 60 shore A, about 30 shore A to about 70 shore A, about 30 shore A to about 80 shore A, about 35 shore A to about 40 shore A, about 35 shore A to about 45 shore A, about 35 shore A to about 50 shore A, about 35 shore A to about 60 shore A, about 35 shore A to about 70 shore A, about 35 shore A to about 80 shore A, about 40 shore A to about 45 shore A, about 40 shore A to about 50 shore A, about 40 shore A to about 60 shore A, about 40 shore A to about 70 shore A, about 40 shore A to about 80 shore A, about 45 shore A to about 50 shore A, about 45 shore A to about 60 shore A, about 45 shore A to about 70 shore A, about 45 shore A to about 80 shore A, about 50 shore A to about 60 shore A, about 50 shore A to about 70 shore A, about 50 shore A to about 80 shore A, about 60 shore A to about 70 shore A, about 60 shore A to about 80 shore A, or about 70 shore A to about 80 shore A. In some embodiments, at least one of the first portion and the second portion have a hardness of about 20 shore A, about 25 shore A, about 30 shore A, about 35 shore A, about 40 shore A, about 45 shore A, about 50 shore A, about 60 shore A, about 70 shore A, or about 80 shore A. In some embodiments, at least one of the first portion and the second portion have a hardness of at least about 20 shore A, about 25 shore A, about 30 shore A, about 35 shore A, about 40 shore A, about 45 shore A, about 50 shore A, about 60 shore A, or about 70 shore A. In some embodiments, at least one of the first portion and the second portion have a hardness of at most about 25 shore A, about 30 shore A, about 35 shore A, about 40 shore A, about 45 shore A, about 50 shore A, about 60 shore A, about 70 shore A, or about 80 shore A. Shore hardness can be measured with a durometer. The hardness value can be determined by the penetration of the durometer indenter foot into a sample of the material (see, e.g., matweb.com/reference/shore-hardness.aspx).

In some embodiments, the first portion and the second portion are formed of the same material. In some embodiments, the first portion and the second portion are formed of the different materials. In some embodiments, at least one of the first portion and the second portion are formed of a biocompatible material. In some embodiments, the biocompatible material is a plastic. In some embodiments, the biocompatible material comprises nylon, silicone, hydrogel, silicone-hydrogel, shell, glass, PMMA, acrylic, metal polycarbonate, polyester, polyethylene, polyethylene terephthalate, polypropylene, polyurethane, polyvinyl chloride, polyvinylidene chloride or any combination thereof.

In some embodiments, the extended period of time is about 0.25 minutes to about 60 minutes. In some embodiments, the extended period of time is about 0.25 minutes to about 0.5 minutes, about 0.25 minutes to about 1 minute, about 0.25 minutes to about 5 minutes, about 0.25 minutes to about 10 minutes, about 0.25 minutes to about 20 minutes, about 0.25 minutes to about 30 minutes, about 0.25 minutes to about 40 minutes, about 0.25 minutes to about 50 minutes, about 0.25 minutes to about 60 minutes, about 0.5 minutes to about 1 minute, about 0.5 minutes to about 5 minutes, about 0.5 minutes to about 10 minutes, about 0.5 minutes to about 20 minutes, about 0.5 minutes to about 30 minutes, about 0.5 minutes to about 40 minutes, about 0.5 minutes to about 50 minutes, about 0.5 minutes to about 60 minutes, about 1 minute to about 5 minutes, about 1 minute to about 10 minutes, about 1 minute to about 20 minutes, about 1 minute to about 30 minutes, about 1 minute to about 40 minutes, about 1 minute to about 50 minutes, about 1 minute to about 60 minutes, about 5 minutes to about 10 minutes, about 5 minutes to about 20 minutes, about 5 minutes to about 30 minutes, about 5 minutes to about 40 minutes, about 5 minutes to about 50 minutes, about 5 minutes to about 60 minutes, about 10 minutes to about 20 minutes, about 10 minutes to about 30 minutes, about 10 minutes to about 40 minutes, about 10 minutes to about 50 minutes, about 10 minutes to about 60 minutes, about 20 minutes to about 30 minutes, about 20 minutes to about 40 minutes, about 20 minutes to about 50 minutes, about 20 minutes to about 60 minutes, about 30 minutes to about 40 minutes, about 30 minutes to about 50 minutes, about 30 minutes to about 60 minutes, about 40 minutes to about 50 minutes, about 40 minutes to about 60 minutes, or about 50 minutes to about 60 minutes. In some embodiments, the extended period of time is about 0.25 minutes, about 0.5 minutes, about 1 minute, about 5 minutes, about 10 minutes, about 20 minutes, about 30 minutes, about 40 minutes, about 50 minutes, or about 60 minutes. In some embodiments, the extended period of time is at least about 0.25 minutes, about 0.5 minutes, about 1 minute, about 5 minutes, about 10 minutes, about 20 minutes, about 30 minutes, about 40 minutes, or about 50 minutes. In some embodiments, the extended period of time is at most about 0.5 minutes, about 1 minute, about 5 minutes, about 10 minutes, about 20 minutes, about 30 minutes, about 40 minutes, about 50 minutes, or about 60 minutes.

Another aspect provided herein is a method for applying a medicament to an eyelid of a patient comprising: applying the medicament to the medicament surface on a first portion and at an intersection of the first portion and a second portion of an eyelid medicament delivery device; inserting the second portion beneath the eyelid of the patient such that the medicament on the medicament surface contacts at least a section of the eyelid of the patient; and removing the second portion from beneath the eyelid of the patient.

In some embodiments, the section of the eyelid of the patient is the lid margin of the eyelid of the patient. In some embodiments, the first portion is generally flat. In some embodiments, the first portion comprises a grip protrusion. In some embodiments, the second portion comprises a distal surface configured to contact an eye of the patient, and a proximal surface configured for contact with an inner eyelid of the patient. In some embodiments, the distal surface has a roughness of less than about 2.5 nm. In some embodiments, the distal surface is configured to conform to the eye of the patient. In some embodiments, the distal surface is concave. In some embodiments, the distal surface has a radius of curvature of about 7 mm to about 18 mm. In some embodiments, the second portion prevents the medicament from contacting the eye of the patient. In some embodiments, at least part of the second portion is covered by an absorbing material. In some embodiments, the method further comprises absorbing the medicament by the absorbing material. In some embodiments, the method further comprise preventing the medicament from contacting the eye of the patient by the absorbing material. In some embodiments, the second portion comprises a sealing protrusion, a sealing recess, or both. In some embodiments, at least one of the sealing protrusion and the sealing recess prevents the medicament from contacting the eye of the patient. In some embodiments, a ratio between a thickness of the second portion protrusion and a height of the sealing protrusion or a depth of the sealing recess is about 10:1 to about 1.5:1. In some embodiments, the second portion comprises a primary second portion and a secondary second portion. In some embodiments, the first portion intersects the second portion at the intersection of the primary second portion and the secondary second portion. In some embodiments, inserting the second portion beneath the eyelid of the patient comprises inserting the primary second portion beneath an upper eyelid of the patient and inserting the secondary second portion beneath a lower eyelid of the patient. In some embodiments, inserting the second portion beneath the eyelid of the patient comprises inserting the second portion beneath an upper eyelid of the patient, inserting the second portion beneath a lower eyelid of the patient, or both. In some embodiments, an outer edge of the second portion is rounded. In some embodiments, the first portion and the second portion are generally coplanar. In some embodiments, the first portion and the second portion are generally perpendicular. In some embodiments, an angle between the first portion and the second portion at the intersection of the first portion and the second portion is about 90 degrees to about 180 degrees. In some embodiments, the medicament surface is coplanar with at least one of the first portion and the second portion. In some embodiments, at least a portion of the medicament surface is perpendicular to at least one of the first portion and the second portion. In some embodiments, the medicament surface is concave. In some embodiments, a thickness of the first portion is greater than or equal to a thickness of the second portion. In some embodiments, a thickness of the first portion is less than or equal to a thickness of the second portion. In some embodiments, a width of the first portion is greater than or equal to a thickness of the second portion. In some embodiments, a width of the first portion is less than or equal to a thickness of the second portion. In some embodiments, a width of at least one of the first portion and the second portion is about 18 mm to about 36 mm. In some embodiments, the second portion has a height of at least about 4 mm. In some embodiments, at least one of the first portion and the second portion has a Young's modulus of about 0.05 MPa to about 10 MPa. In some embodiments, at least one of the first portion and the second portion has a hardness of about 20 shore A to 80 shore A. In some embodiments, the first portion and the second portion are formed of the same material. In some embodiments, the first portion and the second portion are formed of the different materials. In some embodiments, at least one of the first portion and the second portion are formed of a biocompatible material. In some embodiments, the biocompatible material is a plastic. In some embodiments, the biocompatible material comprises nylon, silicone, hydrogel, silicone-hydrogel, shell, glass, PMMA, acrylic, metal polycarbonate, polyester, polyethylene, polyethylene terephthalate, polypropylene, polyurethane, polyvinyl chloride, polyvinylidene chloride or any combination thereof.

Another aspect provided herein is a method for applying a medicament to an eyelid of a patient comprising: receiving an eyelid medicament delivery device having a first portion, a second portion, and the medicament surface on the first portion and at an intersection of the first portion and the second portion, wherein the medicament surface has an applied medicament, an embedded medicament, or both; inserting the second portion beneath the eyelid of the patient such that the medicament on the medicament surface contacts at least a section of the eyelid of the patient; and removing the second portion from beneath the eyelid of the patient.

In some embodiments, the section of the eyelid of the patient is the lid margin of the eyelid of the patient. In some embodiments, the first portion is generally flat. In some embodiments, the first portion comprises a grip protrusion. In some embodiments, the second portion comprises a distal surface configured to contact an eye of the patient, and a proximal surface configured for contact with an inner eyelid of the patient. In some embodiments, the distal surface has a roughness of less than about 2.5 nm. In some embodiments, the distal surface is concave. In some embodiments, the distal surface has a radius of curvature of about 7 mm to about 18 mm. In some embodiments, the second portion prevents the medicament from contacting the eye of the patient. In some embodiments, at least part of the second portion is covered by an absorbing material. In some embodiments, the method further comprises absorbing the medicament by the absorbing material. In some embodiments, the method further comprises preventing the medicament from contacting the eye of the patient by the absorbing material. In some embodiments, the second portion comprises a sealing protrusion, a sealing recess, or both. In some embodiments, at least one of the sealing protrusion and the sealing recess prevents the medicament from contacting the eye of the patient. In some embodiments, a ratio between a thickness of the second portion protrusion and a height of the sealing protrusion or a depth of the sealing recess is about 10:1 to about 1.5:1. In some embodiments, the second portion comprises a primary second portion and a secondary second portion. In some embodiments, the first portion intersects the second portion at the intersection of the primary second portion and the secondary second portion. In some embodiments, inserting the second portion beneath the eyelid of the patient comprises inserting the primary second portion beneath an upper eyelid of the patient and inserting the secondary second portion beneath a lower eyelid of the patient. In some embodiments, inserting the second portion beneath the eyelid of the patient comprises inserting the second portion beneath an upper eyelid of the patient, inserting the second portion beneath a lower eyelid of the patient, or both. In some embodiments, an outer edge of the second portion is rounded. In some embodiments, the first portion and the second portion are generally coplanar. In some embodiments, the first portion and the second portion are generally perpendicular. In some embodiments, an angle between the first portion and the second portion at the intersection of the first portion and the second portion is about 90 degrees to about 180 degrees. In some embodiments, the medicament surface is coplanar with at least one of the first portion and the second portion. In some embodiments, at least a portion of the medicament surface is perpendicular to at least one of the first portion and the second portion. In some embodiments, the medicament surface is concave. In some embodiments, a thickness of the first portion is greater than or equal to a thickness of the second portion. In some embodiments, a thickness of the first portion is less than or equal to a thickness of the second portion. In some embodiments, a width of the first portion is greater than or equal to a thickness of the second portion. In some embodiments, a width of the first portion is less than or equal to a thickness of the second portion. In some embodiments, a width of at least one of the first portion and the second portion is about 18 mm to about 36 mm. In some embodiments, the second portion has a height of at least about 4 mm. In some embodiments, at least one of the first portion and the second portion has a Young's modulus of about 0.05 MPa to about 10 MPa. In some embodiments, at least one of the first portion and the second portion have a hardness of about 20 shore A to 80 shore A. In some embodiments, the first portion and the second portion are formed of the same material. In some embodiments, the first portion and the second portion are formed of the different materials. In some embodiments, at least one of the first portion and the second portion are formed of a biocompatible material. In some embodiments, the biocompatible material is a plastic. In some embodiments, the biocompatible material comprises nylon, silicone, hydrogel, silicone-hydrogel, shell, glass, PMMA, acrylic, metal polycarbonate, polyester, polyethylene, polyethylene terephthalate, polypropylene, polyurethane, polyvinyl chloride, polyvinylidene chloride or any combination thereof.

Another aspect provided herein is a kit for applying a medicament to an eyelid comprising: the device or apparatus herein, and the medicament. In some embodiments, the kit further comprises an instruction sheet, a container, a q-tip, an anesthetic drop, a washing solution, a lid wipe, or any combination thereof.

Another aspect provided herein is an apparatus for applying a medicament to an eye lid margin, comprising: a protector dimensioned to be at least partially insertable between the eye lid and cornea, the protector comprising a barrier portion capable of engaging the interior side of the eye lid; and an applicator extending from the protector and having at least one medicament surface dimensioned to support a strip of medicament in substantial alignment with the eye lid margin when the protector is inserted between the eye lid and cornea, wherein the eye lid margin, the protector, and the applicator combine to define the medicament retaining space that substantially prevents contact of the medicament with the cornea or the interior side of the eye lid outside of the medicament retaining space. In some embodiments, the protector comprises a cornea shield having an interior surface that substantially conforms to an exterior surface of cornea, and an exterior surface that substantially conforms to an interior surface of the eye lid. In some embodiments, the cornea shield comprises an upper portion dimensioned to be insertable between the upper eye lid and the upper portion of the cornea. In some embodiments, the joint between the applicator and the protector is below the upper portion of the cornea shield. In some embodiments, the barrier portion comprises an upper barrier provided on the exterior side of the upper portion of the cornea shield. In some embodiments, the upper barrier is a protrusion extending along the lower end of the upper portion of the cornea shield. In some embodiments, the protrusion is substantially parallel to an upper medicament surface of the applicator. In some embodiments, the barrier portion is integrated to the cornea shield. In some embodiments, the cornea shield comprises a lower portion dimensioned to be insertable between the lower eye lid and the lower portion of the cornea. In some embodiments, the joint between the applicator and the protector is above the lower portion of the cornea shield. In some embodiments, the barrier portion comprises a lower barrier provided on the exterior side of the lower portion of the cornea shield. In some embodiments, the lower barrier is a protrusion extending along the upper end of the lower portion of the cornea shield. In some embodiments, the protrusion is substantially parallel to a lower medicament surface of the applicator. In some embodiments, the barrier portion is integrated to the cornea shield. In some embodiments, the cornea shield comprises an upper portion dimensioned to be insertable between the upper eye lid and the upper portion of the cornea, and a lower portion dimensioned to be insertable between the lower eye lid and the lower portion of the cornea. In some embodiments, the joint between the applicator and the protector is between the upper portion and lower portion of the cornea shield. In some embodiments, the barrier portion comprises an upper barrier provided on the exterior side of the upper portion of the cornea shield, and a lower barrier provided on the exterior side of the lower portion of the cornea shield. In some embodiments, the upper barrier is a protrusion extending along the lower end of the upper portion of the cornea shield. In some embodiments, the protrusion is substantially parallel to an upper medicament surface of the applicator. In some embodiments, the lower barrier is a protrusion extending along the upper end of the lower portion of the cornea shield. In some embodiments, the protrusion is substantially parallel to a lower medicament surface of the applicator. In some embodiments, the barrier portion further comprises two side barriers provided on the exterior side of the cornea shield connecting the upper and lower barriers. In some embodiments, the barrier portion is integrated to the cornea shield. In some embodiments, the plate comprises a distal portion that connects with the protector and a proximal portion dimensioned for finger grip. In some embodiments, the medicament surface is provided on the distal portion of the plate. In some embodiments, at least one surface irregularity is provided at the proximal portion of the plate to facilitate finger grip. In some embodiments, the surface irregularities comprise at least one ridge rising from the surface of the plate. In some embodiments, the applicator is integrated with the protector. In some embodiments, the protector comprises a cornea shield dimensioned to be insertable between the eye lid and the cornea, the cornea shield extending between a distal end and a proximal end. In some embodiments, the barrier portion comprises lateral barrier provided on the exterior side the cornea shield. In some embodiments, the lateral barrier is a protrusion extending along proximal end of the cornea shield. In some embodiments, the protrusion is substantially parallel to the medicament surface of the applicator. In some embodiments, the barrier portion is integrated to the cornea shield. In some embodiments, the applicator comprises a plate extending from the proximal end of the cornea shield. In some embodiments, the plate comprises a distal portion that joins the proximal end of protector, and a proximal portion dimensioned for finger grip. In some embodiments, the medicament surface is provided on the distal portion of the plate. In some embodiments, at least one surface irregularity is provided at the proximal portion of the plate to facilitate finger grip. In some embodiments, the surface irregularities comprise at least one ridge rising from the surface of the plate. In some embodiments, the applicator further comprises two side barriers protruding from the plate close to the lateral ends of the medicament surface. In some embodiments, the side barriers of the applicator are connected to the two ends of the lateral barrier of the protector extending from the end of the lateral barrier. In some embodiments, the side barriers protrude from the plate of the applicator at a height greater than that of the lateral barrier protruding from the cornea shield. In some embodiments, the applicator is integrated with the protector. In some embodiments, the protector comprises an elongated body laterally extending between two ends and dimensioned to be insertable between the eye lid and the cornea. In some embodiments, the barrier portion is the exterior surface of the elongated body that extends from the joint between the applicator and the protector to where the elongated body engages the inner side of the eye lid. In some embodiments, the applicator comprises a plate having top and bottom sides. In some embodiments, the applicator extends from an upper portion of elongated body, and wherein the medicament surface is provided on the bottom side of the plate along the joint between the applicator and the protector. In some embodiments, the applicator extends from a lower portion of the elongated body, and wherein the medicament surface is provided on the top side of the plate along the joint between the applicator and the protector. In some embodiments, the elongated body is made of a material capable of absorbing the medicament. In some embodiments, the protector comprises an elongated body laterally extending between two ends, the elongated body having an upper portion dimensioned to be insertable between the upper eye lid and the cornea and a lower portion dimensioned to be insertable between the lower eye lid and the cornea. In some embodiments, the barrier portion is the exterior surface of the elongated body that extends from the joint between the applicator and the protector to where the elongated body engages the inner sides of the eye lids. In some embodiments, the applicator comprises a plate having top and bottom sides. In some embodiments, the applicator extends from between the upper and lower portions of elongated body, and wherein the medicament surface is provided on both sides of the plate along the joint between the applicator and the protector. In some embodiments, the elongated body is made of a material capable of absorbing the medicament.

Another aspect provided herein is a method of applying a medicament to an eye lid margin, the method comprising: inserting a protector between the eye lid and the cornea; and contacting the eye lid margin with the medicament provided on an applicator, wherein the eye lid margin, the protector, and the applicator combine to define a medicament retaining space that substantially prevents contact of the medicament with the cornea or the interior side of the eye lid outside of the medicament retaining space.

In some embodiments, the protector and the applicator are joined together. In some embodiments, the protector comprises a cornea shield having an interior surface that substantially conforms to an exterior surface of cornea, and an exterior surface that substantially conforms to an interior surface of the eye lid. In some embodiments, the cornea shield comprises an upper portion dimensioned to be insertable between the upper eye lid and the upper portion of the cornea. In some embodiments, the joint between the applicator and the protector is below the upper portion of the cornea shield. In some embodiments, the barrier portion comprises an upper barrier provided on the exterior side of the upper portion of the cornea shield. In some embodiments, the upper barrier is a protrusion extending along the lower end of the upper portion of the cornea shield. In some embodiments, the protrusion is substantially parallel to an upper medicament surface of the applicator. In some embodiments, the barrier portion is integrated to the cornea shield. In some embodiments, the cornea shield comprises a lower portion dimensioned to be insertable between the lower eye lid and the lower portion of the cornea. In some embodiments, the joint between the applicator and the protector is above the lower portion of the cornea shield. In some embodiments, the barrier portion comprises a lower barrier provided on the exterior side of the lower portion of the cornea shield. In some embodiments, the lower barrier is a protrusion extending along the upper end of the lower portion of the cornea shield. In some embodiments, the protrusion is substantially parallel to a lower medicament surface of the applicator. In some embodiments, the barrier portion is integrated to the cornea shield. In some embodiments, the cornea shield comprises an upper portion dimensioned to be insertable between the upper eye lid and the upper portion of the cornea, and a lower portion dimensioned to be insertable between the lower eye lid and the lower portion of the cornea. In some embodiments, the joint between the applicator and the protector is between the upper portion and lower portion of the cornea shield. In some embodiments, the barrier portion comprises an upper barrier provided on the exterior side of the upper portion of the cornea shield, and a lower barrier provided on the exterior side of the lower portion of the cornea shield. In some embodiments, the upper barrier is a protrusion extending along the lower end of the upper portion of the cornea shield. In some embodiments, the protrusion is substantially parallel to an upper medicament surface of the applicator. In some embodiments, the lower barrier is a protrusion extending along the upper end of the lower portion of the cornea shield. In some embodiments, the protrusion is substantially parallel to a lower medicament surface of the applicator. In some embodiments, the barrier portion further comprises two side barriers provided on the exterior side of the cornea shield connecting the upper and lower barriers. In some embodiments, the barrier portion is integrated to the cornea shield. In some embodiments, the plate comprises a distal portion that connects with the protector and a proximal portion dimensioned for finger grip. In some embodiments, the medicament surface is provided on the distal portion of the plate. In some embodiments, at least one surface irregularity is provided at the proximal portion of the plate to facilitate finger grip. In some embodiments, the surface irregularities comprise at least one ridge rising from the surface of the plate. In some embodiments, the applicator is integrated with the protector. In some embodiments, the protector comprises a cornea shield dimensioned to be insertable between the eye lid and the cornea, the cornea shield extending between a distal end and a proximal end. In some embodiments, the barrier portion comprises lateral barrier provided on the exterior side the cornea shield. In some embodiments, the lateral barrier is a protrusion extending along proximal end of the cornea shield. In some embodiments, the protrusion is substantially parallel to the medicament surface of the applicator. In some embodiments, the barrier portion is integrated to the cornea shield. In some embodiments, the applicator comprises a plate extending from the proximal end of the cornea shield. In some embodiments, the plate comprises a distal portion that joins the proximal end of protector, and a proximal portion dimensioned for finger grip. In some embodiments, the medicament surface is provided on the distal portion of the plate. In some embodiments, at least one surface irregularity is provided at the proximal portion of the plate to facilitate finger grip. In some embodiments, the surface irregularities comprise at least one ridge rising from the surface of the plate. In some embodiments, the applicator further comprises two side barriers protruding from the plate close to the lateral ends of the medicament surface. In some embodiments, the side barriers of the applicator are connected to the two ends of the lateral barrier of the protector extending from the end of the lateral barrier. In some embodiments, the side barriers protrude from the plate of the applicator at a height greater than that of the lateral barrier protruding from the cornea shield. In some embodiments, the applicator is integrated with the protector. In some embodiments, the protector comprises an elongated body laterally extending between two ends and dimensioned to be insertable between the eye lid and the cornea. In some embodiments, the barrier portion is the exterior surface of the elongated body that extends from the joint between the applicator and the protector to where the elongated body engages the inner side of the eye lid. In some embodiments, the applicator comprises a plate having top and bottom sides. In some embodiments, the applicator extends from an upper portion of elongated body, and wherein the medicament surface is provided on the bottom side of the plate along the joint between the applicator and the protector. In some embodiments, the applicator extends from a lower portion of the elongated body, and wherein the medicament surface is provided on the top side of the plate along the joint between the applicator and the protector. In some embodiments, the elongated body is made of a material capable of absorbing the medicament. In some embodiments, the protector comprises an elongated body laterally extending between two ends, the elongated body having an upper portion dimensioned to be insertable between the upper eye lid and the cornea and a lower portion dimensioned to be insertable between the lower eye lid and the cornea. In some embodiments, the barrier portion is the exterior surface of the elongated body that extends from the joint between the applicator and the protector to where the elongated body engages the inner sides of the eye lids. In some embodiments, the applicator comprises a plate having top and bottom sides. In some embodiments, the applicator extends from between the upper and lower portions of elongated body, and wherein the medicament surface is provided on both sides of the plate along the joint between the applicator and the protector. In some embodiments, the elongated body is made of a material capable of absorbing the medicament.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 is a cross-sectional illustration of a first exemplary device for applying a medicament to an eyelid of a patient, per some embodiments herein;

FIG. 2 is a cross-sectional illustration of a second exemplary device for applying a medicament to an eyelid of a patient, per some embodiments herein;

FIG. 3 is a cross-sectional illustration of a third exemplary device for applying a medicament to an eyelid of a patient, per some embodiments herein;

FIG. 4 is a cross-sectional illustration of a fourth exemplary device for applying a medicament to an eyelid of a patient, per some embodiments herein;

FIG. 5 is a cross-sectional illustration of a fifth exemplary device for applying a medicament to an eyelid of a patient, per some embodiments herein;

FIG. 6 is a cross-sectional illustration of a sixth exemplary device for applying a medicament to an eyelid of a patient, per some embodiments herein;

FIG. 7 is a cross-sectional illustration of a seventh exemplary device for applying a medicament to an eyelid of a patient, per some embodiments herein;

FIG. 8 is a cross-sectional illustration of an eighth exemplary device for applying a medicament to an eyelid of a patient, per some embodiments herein;

FIG. 9 is another cross-sectional illustration of the third exemplary device for applying a medicament to an eyelid of a patient, per some embodiments herein;

FIG. 10 is a top-front perspective illustration of the third exemplary device for applying a medicament to an eyelid of a patient, per some embodiments herein;

FIG. 11 is a top-right-back perspective illustration of the third exemplary device for applying a medicament to an eyelid of a patient, per some embodiments herein;

FIG. 12 is another cross-sectional illustration of the fourth exemplary device for applying a medicament to an eyelid of a patient, per some embodiments herein;

FIG. 13 is a top-front perspective illustration of the fourth exemplary device for applying a medicament to an eyelid of a patient, per some embodiments herein;

FIG. 14 is a top-right-back perspective illustration of the fourth exemplary device for applying a medicament to an eyelid of a patient, per some embodiments herein;

FIG. 15 is another cross-sectional illustration of the sixth exemplary device for applying a medicament to an eyelid of a patient, per some embodiments herein;

FIG. 16 is a top view illustration of the sixth exemplary device for applying a medicament to an eyelid of a patient, per some embodiments herein;

FIG. 17 is a top-front-left perspective view illustration of the sixth exemplary device for applying a medicament to an eyelid of a patient in the eye of a patient, per some embodiments herein;

FIG. 18 is a top-front-left perspective view illustration of the sixth exemplary device with hidden lines shown for applying a medicament to an eyelid of a patient in the eye of a patient, per some embodiments herein;

FIG. 19A is a front-top perspective view illustration of the ninth exemplary device being inserted into an eyelid of a patient per some embodiments herein;

FIG. 19B, is a front-left perspective view illustration of a ninth exemplary device for applying a medicament to an eyelid of a patient, per some embodiments herein;

FIG. 20A is a cross-sectional illustration of a tenth exemplary device for applying a medicament to an eyelid of a patient, per some embodiments herein; and

FIG. 20B is a cross-sectional illustration of a tenth exemplary device for applying a medicament to an eyelid of a patient, per some embodiments herein.

FIG. 21 is a cross-sectional illustration of an eleventh exemplary device for applying a medicament to an eyelid of a patient, per some embodiments herein.

FIG. 22 is a cross-sectional illustration of a twelfth exemplary device for applying a medicament to an eyelid of a patient, per some embodiments herein.

FIG. 23 is a cross-sectional illustration of a thirteenth exemplary device for applying a medicament to an eyelid of a patient, per some embodiments herein.

FIG. 24A is a front-top perspective view of a fourteenth exemplary device for applying a medicament to an eyelid of a patient, per some embodiments herein.

FIG. 24B is a side perspective view of a fourteenth exemplary device for applying a medicament to an eyelid of a patient, per some embodiments herein.

FIG. 25A is a front-top perspective view of a fifteenth exemplary device for applying a medicament to an eyelid of a patient, per some embodiments herein.

FIG. 25B is a top perspective of a fifteenth exemplary device for applying a medicament to an eyelid of a patient, per some embodiments herein.

FIG. 26 illustrates a cross-sectional schematic of an exemplary normally functioning eyelid in relation to an ocular surface.

### DETAILED DESCRIPTION

Some eyelid disorders are treated by applying a medicament to the eyelid, e.g. eyelid margin, for one or more therapeutic sessions. In some embodiments, the medicament is a liquid, a solid, or a semi-solid (e.g. gel, ointment, and paste). For example, some treatment of MGD involves the application of a medicament containing a keratolytic agent to the eyelid, e.g. eyelid margin. In some instances, the application involves maintaining contact between the medicament and eyelid margin for an extended period of time (e.g. at least 0.25 min, 0.5 min 1 min, at least 5 min, at least 10 min, at least 15 min, at least 20 min, at least 25 min, at least 30 min, at least 40 min, at least 50 min, at least 1 hour, at least 1.5 hours, at least 2 hours, etc.). Accordingly, the device and method disclosure herein, in some embodiments, allows the medicament to remain in contact with the eyelid margin for an extended period of time without additional support (e.g. held in place by hand or by a separate holding apparatus). In some instances, the medicaments are harmful to other ocular tissues such as the cornea. Accordingly, the device and method disclosure herein, in some embodiments, allows application of such medicaments to the eyelid, e.g. eyelid margin, of a patient while protecting the adjunct ocular tissues such as the cornea.

### Certain Terms and Definitions

Unless otherwise defined, all technical terms used herein have the same meaning as commonly understood by one of ordinary skill in the art to which this disclosure belongs.

As used herein, the singular forms “a,” “an,” and “the” include plural references unless the context clearly dictates otherwise. Any reference to “or” herein is intended to encompass “and/or” unless otherwise stated.

As used herein, the term “about” in some cases refers to an amount that is approximately the stated amount.

As used herein, the term “about” refers to an amount that is near the stated amount by 10%, 5%, or 1%, including increments therein.

As used herein, the term “about” in reference to a percentage refers to an amount that is greater or less the stated percentage by 10%, 5%, or 1%, including increments therein.

As used herein, the term “generally” in some cases refers to an amount that is approximately the stated amount. In some cases, the term “generally” refers to an amount that is near the stated amount by 10%, 5%, or 1%, including increments therein.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

### Devices for Applying a Medicament to an Eyelid

Provided herein is a device for applying a medicament to an eyelid. In some embodiments, the device comprises a first portion having a distal end and a proximal end. In some embodiments, the first portion comprises a medicament surface close to the distal end. In some embodiments, the first portion is shaped and dimensioned to allow close contact between an eye lid margin and the medicament surface for an extended period of time without any additional support provided on the first portion proximal to the medicament surface. In some embodiments, the medicament surface is within 1 mm, 2 mm, 3 mm, 4 mm, 5 mm, 6 mm, 7 mm, 8 mm, 9 mm, 10 mm, or more including increments from the distal end. In some embodiments, the device comprises an energy source. In some embodiments, the energy source can aid with the activity of a medicament through the application of warmth to enhance drug penetration, activate the drug or melt blockages within gland orifices, etc.

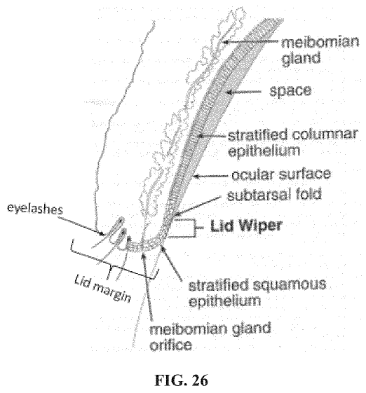

FIG. 26 illustrates a schematic of a portion of an exemplary eye surface and lid. As illustrated in the figure, at the end of the lid, eye lashes can be observed. Moving inward from the lashes, the inner surface of the lid comprises a stratified squamous epithelium region located proximal to the lashes. Further along the inner surface of the lid, the stratified squamous epithelium leads into the lid wiper region, which is the region of the inner surface that comes into contact with the ocular surface (or contact lens) (e.g., in a normally functioning eyelid). Moving from the lid wiper region (e.g., moving along the inner surface of the lid in a direction distal to the lashes), the inner surface of the lid comprises a subtarsal fold region and a stratified columnar epithelium region. In some instances, a palpebra conjunctiva extends over all or a portion of the inner surface of the lid, such as having a leading edge in the lid wiper region.

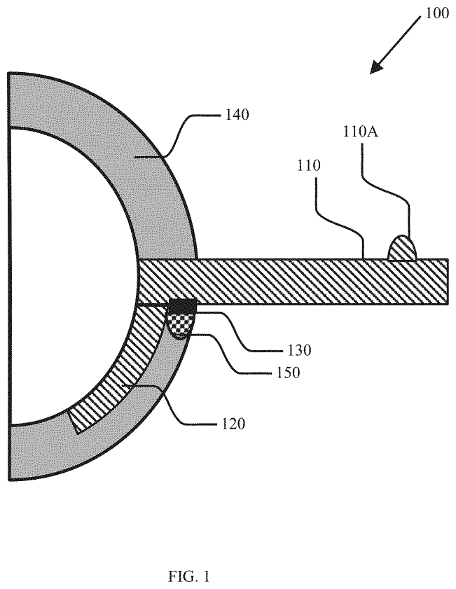

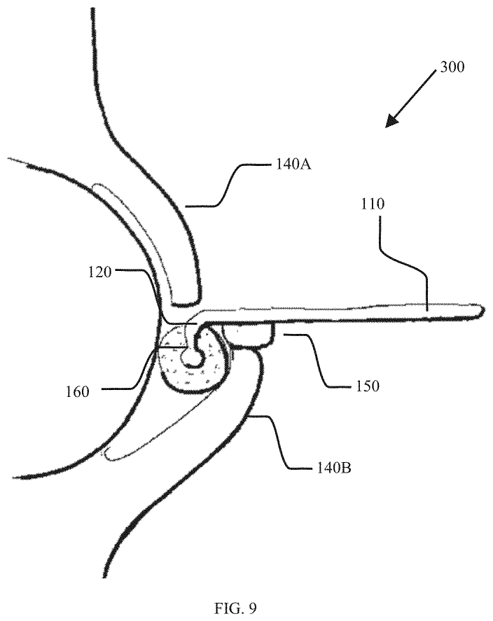

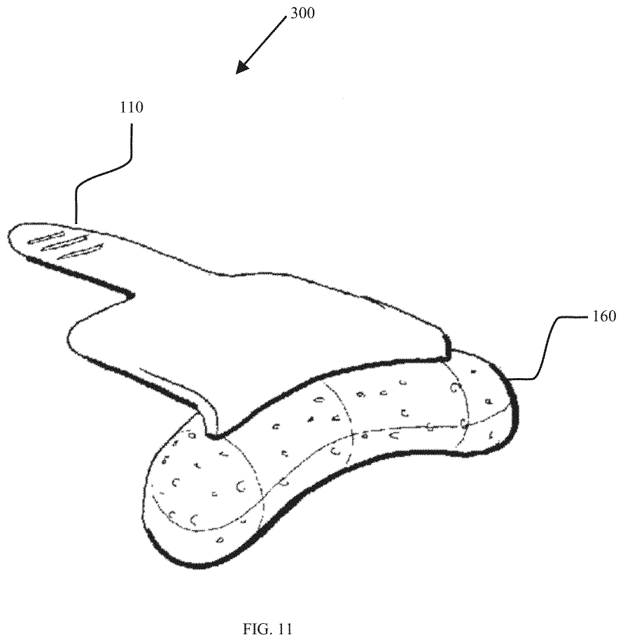

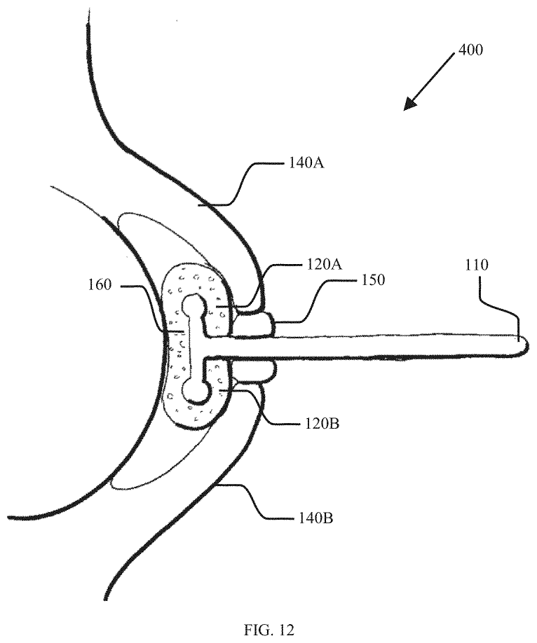

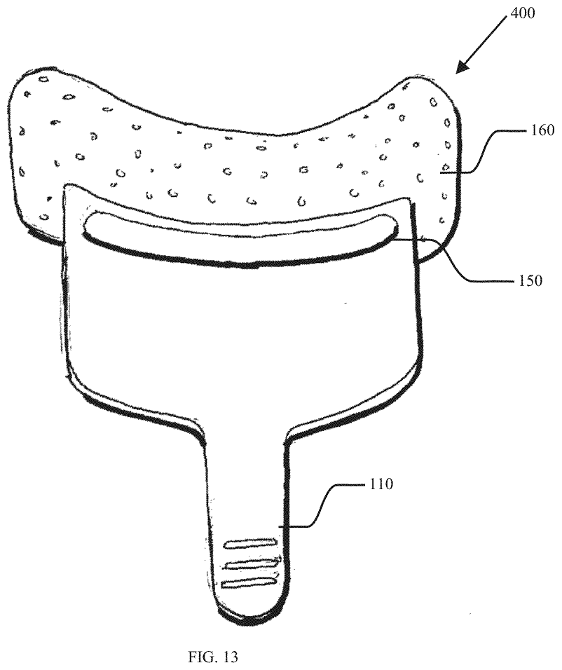

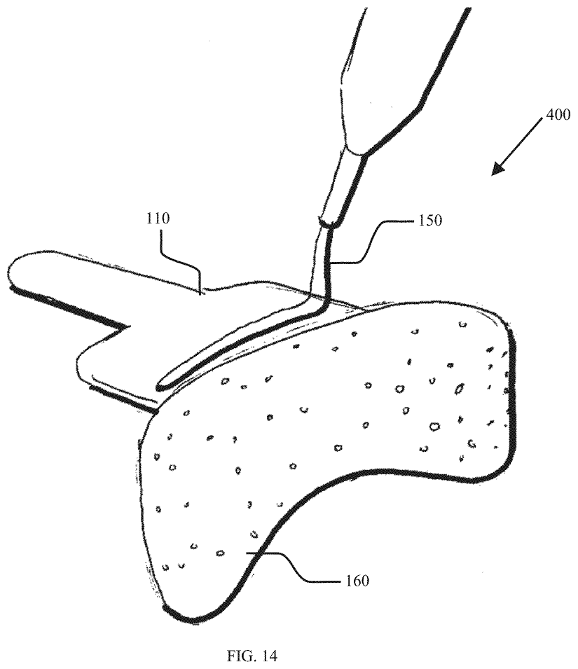

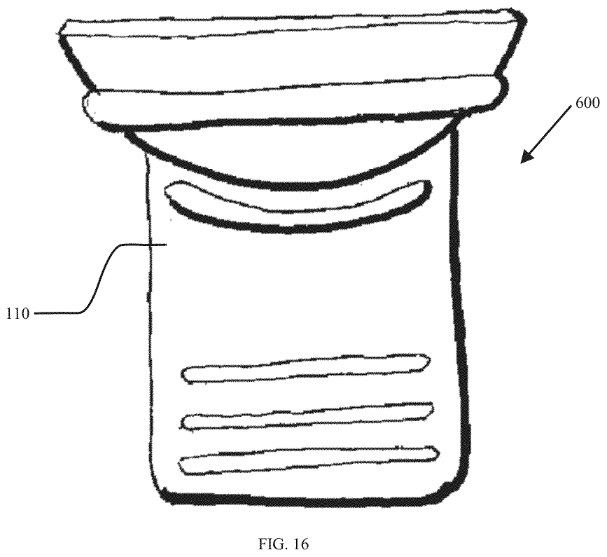

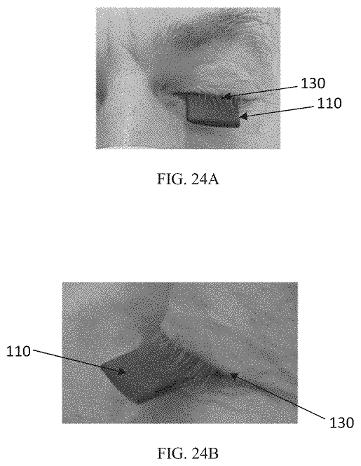

Also provided herein as non-limiting examples, per FIGS. 1-24B are first, second, third, fourth, fifth, sixth, seventh, eighth, ninth, tenth, eleventh and twelfth devices 100 200 300 400 500 600 700 800 900 1000 1100 1200 for applying a medicament 150 to an eyelid 140 of a patient. As shown therein, the devices 100 200 300 400 500 600 700 800 900 1000 1100 1200 comprise a first portion 110, a second portion 120, and a medicament surface 130.

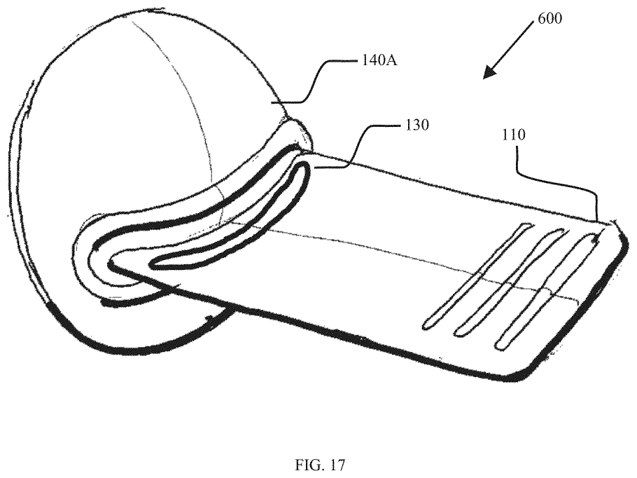

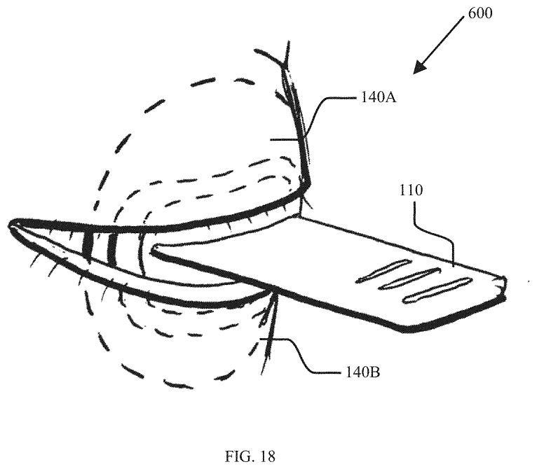

In some embodiments, the first portion 110 is shaped and dimensioned to allow close contact between an eye lid margin and the medicament surface 130. In some embodiments, the first portion 110 is shaped and dimensioned to allow close contact between an eye lid margin and the medicament surface 130 for an extended period of time. In some embodiments, the first portion 110 is shaped and dimensioned to allow close contact between an eye lid margin and the medicament surface 130 for an extended period of time without any additional support provided on the first portion 110 proximal to the medicament surface 130. In some embodiments, the extended period of time is a sufficient time for the medicament 150 to affect the meibomian glands of the eye of the patient. One of skill in the art would immediately recognize the sufficient times to affect the meibomian glands for different medicaments 150. In some embodiments, the first portion 110 and/or the second portion 120 are shaped and dimensioned to allow insertion of the second portion 120 between the eye and eyelid 140 of a patient using only one hand. In some embodiments, the first portion 110 and/or the second portion 120 are shaped and dimensioned to allow the second portion 120 to maintain its position between the eye and eyelid 140 of a patient without physical intervention. In some embodiments, per FIGS. 17 and 18 the second portion 120 is configured to be inserted beneath an upper eyelid 140A of the patient, a lower eyelid 140B of the patient, or both. As contemplated herein, the device and method of the present disclosure may (1) comprise or utilize the first (applicator) portion without the second (protector) portion; (2) comprise or utilize the first (applicator) portion and the second (protector) portion as separate components of a kit; or (3) comprise or utilize the first (applicator) portion and the second (protector) portion as integrated components of a device or apparatus.

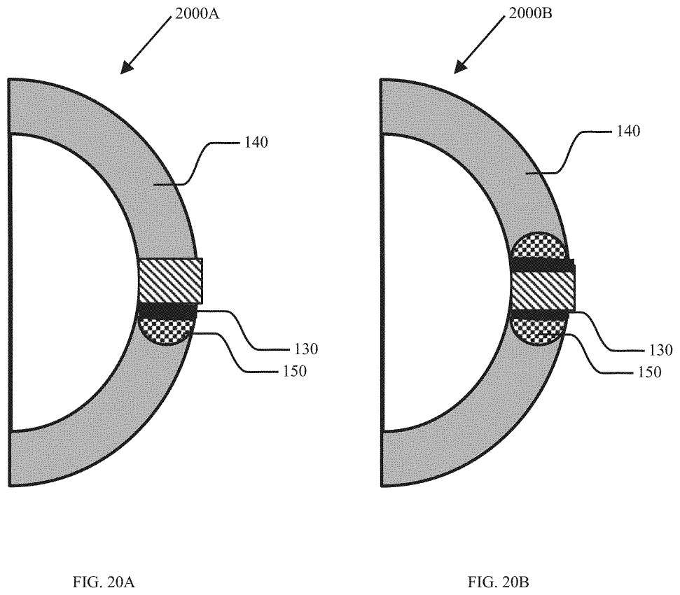

Also provided herein per FIGS. 20A-B are tenth devices 2000A 2000B for applying a medicament 150 to an eyelid 140 comprising a medicament surface 130 configured to absorb or contain the medicament 150, wherein the medicament surface 130 contacts at least a section of the eyelid 140 of the patient. In some embodiments, per FIG. 20A, the tenth device 2000A comprises one medicament surface 130 configured to absorb or contain the medicament. 150 and apply medicament 150 to an upper eyelid 140 or a lower eyelid 140 of a patient. In some embodiments, per FIG. 20B, the tenth device 2000B comprises two opposing medicament surface 130 configured to absorb or contain the medicament 150 configured to absorb or contain the medicament. 150 and apply medicament 150 to an upper eyelid 140 and a lower eyelid 140 of a patient.

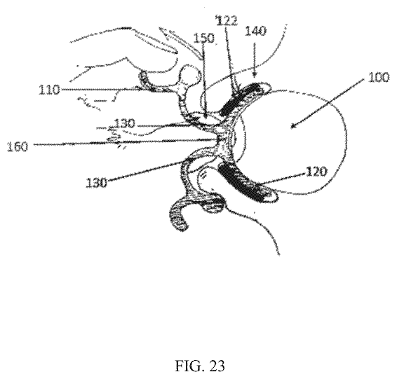

In some embodiments, the medicament is applied to the eyelid and kept separate from the ocular surface. In some embodiments the ocular surface is protect by a device (e.g., a shield) as described herein. Also provided herein per FIG. 23 is a thirteenth device for applying a medicament 150 to an eyelid 140 comprising a medicament surface 130 configured to absorb or contain the medicament 150, wherein the medicament surface 130 contacts at least a region of the eyelid 140 of the patient. In some embodiments the thirteenth device comprises a shield 160 with a sealing element 122. In some embodiments, the sealing elements 122 are attached directly to the first portion without the shield. In some embodiments, the thirteenth device comprises the sealing elements 122 and does not comprise the shield 160. In some embodiments, the device comprises a corneal protection element 180 attached to the first portion, without the shield 160 or sealing elements 122. In some embodiments, a user can provide a force on the first element 110 to the lower lid to provide a tighter seal on the lower lid (e.g., in the z-axis as shown in FIG. 23). In some embodiments the element 110 has spring properties so that when released over the eyelid it creates pressure between 110 and 122 that would further seal the medicament 150 within surface 130.

In some embodiments, the shield 160 is used in conjunction with a medicament that is not compatible with the ocular surface. For example, the device can be used to apply keratolytic agents that require physical separation from the ocular surface (see, e.g., FIG. 23). In some embodiments, the device physically separates the eyelid from the ocular surface using a shield 160 and allows for the treatment of the eyelid separate from the ocular surface. The medicament can comprise salicylic acid or urea at high concentrations. For example, the medicament can comprise salicylic acid or urea at high concentrations for the treatment of meibomian gland disorder and lid wiper epitheliopathy. The medicament can comprise tea tree oil at high concentrations (e.g. 10 weight % to 50 weight % or above 1%). The medicament can comprise tea tree oil at high concentrations to treat demodex such as 10 weight % to 50 weight %. The medicament can comprise therapeutic agents that are not tolerable on an ocular surface at a high concentration.

### First (Applicator) and Second (Protector) Portions

In some embodiments, the first portion 110 has a distal end and a proximal end. In some embodiments, the first portion 110 is generally flat. In some embodiments, the first portion 110 is generally planar. In some embodiments, a bisecting plane of the first portion 110 is generally flat. In some embodiments, a bisecting plane of the first portion 110 is generally planar.

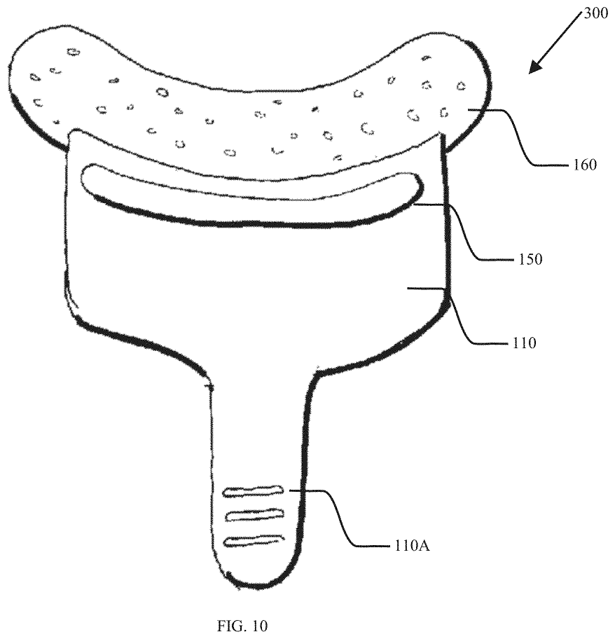

As shown in FIGS. 1 and 10 the first portion 110 comprises a grip protrusion 110A. In some embodiments, the first portion 110 comprises two or more grip protrusions 110A. In some embodiments, one surface of the first portion 110 comprises the grip protrusion 110A. In some embodiments, two or more surfaces of the first portion 110 comprise the grip protrusion 110A. In some embodiments, the grip protrusion 110A is configured to increase the friction of the first portion 110. In some embodiments, the grip protrusion 110A is configured to improve the grip and/or stability of the device in the hands of a caregiver during use.

In some embodiments, the second portion 120 is configured to be inserted beneath an eyelid 140 of a patient. In some embodiments, the second portion 120 is configured to be inserted beneath an upper eyelid 140A of the patient, a lower eyelid 140B of the patient, or both. In some embodiments, the second portion 120 has a sufficiently low roughness to enable its insertion beneath the eyelid 140 of the patient. In some embodiments, the distal surface has a roughness of less than about 2.5 nm. One of ordinary skill in the art will immediately appreciate a sufficiently low roughness to prevent abrasion and/or damage to the eye of the patient.

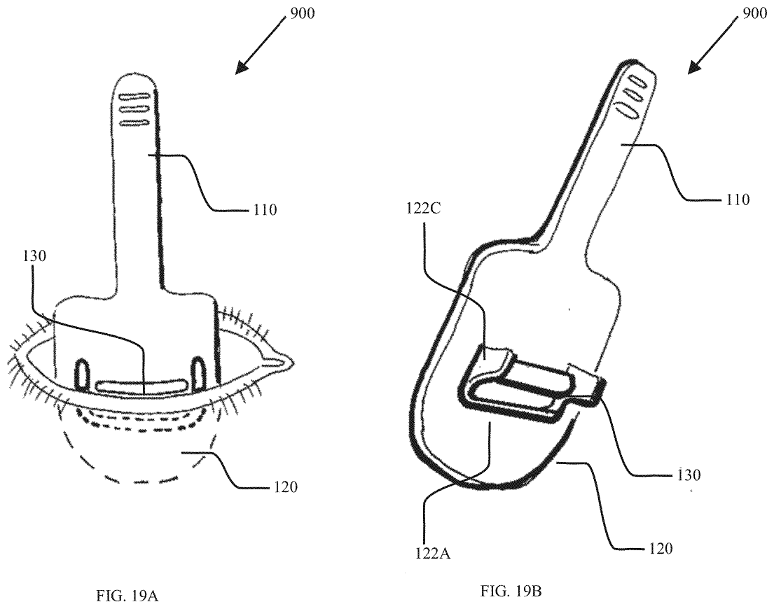

In some embodiments, per FIGS. 19A-B the first portion 110 and the second portion 120 are generally coplanar. In some embodiments, the first portion 110 and the second portion 120 are generally perpendicular. In some embodiments, an angle between the first portion 110 and the second portion 120 at the intersection of the first portion 110 and the second portion 120 is about 90 degrees to about 180 degrees.

In some embodiments, the second portion 120 comprises a distal surface configured to contact an eye of the patient. In some embodiments, the distal surface has a sufficiently low roughness to enable the insertion of the second portion 120 beneath the eyelid 140 of the patient. In some embodiments, the distal surface has a roughness of less than about 2.5 nm. In some embodiments, the distal surface is concave. In some embodiments, the distal surface has a radius of curvature of about 7 mm to about 18 mm. In some embodiments, the distal surface conforms to the eye of the patient. In some embodiments, at least one of the roughness, the concavity, or the radius of curvature of the distal surface allows it to conform to the eye of the patient.

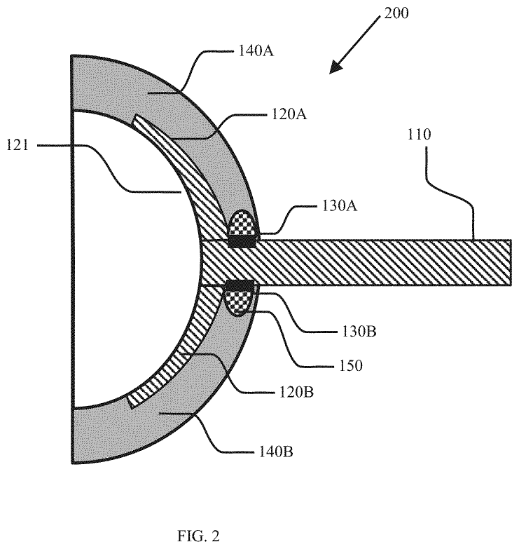

In some embodiments, per FIG. 2, the second portion 120 comprises a distal surface 121 configured for contact with an inner eyelid 140 of the patient. In some embodiments, the distal surface 121 has a sufficiently low roughness to enable the insertion of the second portion 120 beneath the eyelid 140 of the patient. In some embodiments, the distal surface 121 has a roughness of less than about 2.5 nm. In some embodiments, the distal surface 121 is convex. In some embodiments, the distal surface 121 has a radius of curvature of about 7 mm to about 18 mm. In some embodiments, the distal surface 121 conforms to the eyelid 140 of the patient. In some embodiments, at least one of the roughness, the convexity, or the radius of curvature of the distal surface 121 allows it to conform to the eyelid 140 of the patient.

In some embodiments, a thickness of the first portion 110 is greater than or equal to a thickness of the second portion 120. In some embodiments, a thickness of the first portion 110 is less than or equal to a thickness of the second portion 120. In some embodiments, a width of the first portion 110 is greater than or equal to a thickness of the second portion 120. In some embodiments, a width of the first portion 110 is less than or equal to a thickness of the second portion 120. In some embodiments, a width of at least one of the first portion 110 and the second portion 120 is about 18 mm to about 36 mm. In some embodiments, the second portion 120 has a height of at least about 4 mm. In some embodiments, at least one of the width of the first portion 110, the thickness of the first portion 110, the width of the second portion 120, or the thickness of the second portion 120, are measured as a minimum dimension, a maximum dimension, or an average dimension.

In some embodiments, at least one of the width of the first portion 110 and the thickness of the first portion 110 allow the first portion 110 to have a sufficient rigidity to maneuver the second portion 120 under the eyelid 140 of the patient. In some embodiments, at least one of the width of the first portion 110 and the thickness of the first portion 110 enables a sufficient durability to withstand forces applied thereon during the insertion of the second portion 120 under the eyelid 140 of the patient. In some embodiments, at least one of the width of the second portion 120, or the thickness of the second portion 120 allow the second portion 120 to have a sufficient rigidity to be inserted under the eyelid 140 of the patient. In some embodiments, at least one of the width of the second portion 120 or the thickness of the second portion 120 enables a sufficient durability to withstand forces applied thereon during its insertion under the eyelid 140 of the patient. In some embodiments, at least one of the width of the second portion 120 and the thickness of the first portion 110 enables a sufficient flexibility to conform to the surface of the eyelid 140 of the patient.

In some embodiments, at least one of the first portion 110 and the second portion 120 has a Young's modulus of about 0.05 MPa to about 10 MPa. In some embodiments, the Young's modulus of at least one of the first portion 110 and the second portion 120 enables a sufficient rigidity to maneuver the second portion 120 under the eyelid 140 of the patient. In some embodiments, the Young's modulus of at least one of the first portion 110 and the second portion 120 enables a sufficient durability to withstand forces applied thereon during the insertion of the second portion 120 under the eyelid 140 of the patient. In some embodiments, the Young's modulus of the second portion 120 enables a sufficient flexibility to conform to the surface of the eyelid 140 of the patient.

In some embodiments, at least one of the first portion 110 and the second portion 120 have a hardness of about 20 shore A to 80 shore A. In some embodiments, the hardness of at least one of the first portion 110 and the second portion 120 enables a sufficient rigidity to maneuver the second portion 120 under the eyelid 140 of the patient. In some embodiments, the hardness of at least one of the first portion 110 and the second portion 120 enables a sufficient durability to withstand forces applied thereon during the insertion of the second portion 120 under the eyelid 140 of the patient. In some embodiments, the hardness of the second portion 120 enables a sufficient flexibility to conform to the surface of the eyelid 140 of the patient. In some embodiments, the hardness of the second portion 120 enables a sufficient flexibility to conform to the surface of the eyelid 140 of the patient without damaging the eyelid 140 or the eye of the patient.

One of ordinary skill in the art will immediately recognize the metes and bounds of a sufficient rigidity and durability for insertion of the second portion 120 under the eyelid 140 of the patient. One of ordinary skill in the art will immediately recognize the metes and bounds of a sufficient flexibility for conformity to the surface of the eyelid 140 of the patient and/or for preventing damage to the patient's eyelid 140 or eye.

In some embodiments, the first portion 110 and the second portion 120 are formed of the same material. In some embodiments, the first portion 110 and the second portion 120 are formed of the different materials. In some embodiments, at least one of the first portion 110 and the second portion 120 are formed of a biocompatible material. In some embodiments, the biocompatible material is a plastic. In some embodiments, the biocompatible material comprises nylon, silicone, hydrogel, silicone-hydrogel, shell, Glass, PMMA, Acrylic, Metal polycarbonate, polyester, polyethylene, polyethylene terephthalate, polypropylene, polyurethane, polyvinyl chloride, polyvinylidene chloride or any combination thereof. In some embodiments, the first portion 110 and the second portion 120 are formed of any material having the aforementioned Young's modulus and/or hardness. In some embodiments, the first portion 110 and the second portion 120 are formed of any material capable of contacting the eye and/or eyelid 140 of the patient without damage or contamination thereof. In some embodiments, at least one of the first portion 110 and the second portion 120 are formed of a material that is impermeable to the medicament 150. In some embodiments, at least one of the first portion 110 and the second portion 120 are formed of a material that is impermeable to the medicament 150 to prevent the medicament 150 from contacting the eye of the patient. In some embodiments, at least a portion of one or more of the first portion 110 and the second portion 120 are coated with a material that is impermeable to the medicament 150. In some embodiments, at least a portion of one or more of the first portion 110 and the second portion 120 are coated with a material that is impermeable to the medicament 150 to prevent the medicament 150 from contacting the eye of the patient. In some embodiments, the medicament surface 130 is coated with a material that is permeable to the medicament 150. As such, in some embodiments, the material of at least one of the first portion 110 and the second portion 120 is determined by the fluidic characteristics of specific medicament 150.

### Primary and Secondary Second Portions and Medicament Surfaces

As shown in FIGS. 1, 3, 5, 7, 9-11 and 22 the second portion 120 comprises only a primary second portion. As shown in FIGS. 2, 4, 6, 8, and 12-18 the second portion 120 comprises a primary second portion 120A and a secondary second portion 120B. In some embodiments, the second portion 120 does not comprise the secondary second portion 120B. As used herein, the “primary” and “secondary” designation of the secondary portion refers to the different areas that the secondary portion intends to protection, e.g. upper cornea or lower cornea, instead of referring to their functional hierarchy.

In some embodiments, the first portion 110 intersects the second portion 120 at the intersection of the primary second portion 120A and the secondary second portion 120B. In some embodiments, an outer edge of the primary second portion 120A, the secondary second portion 120B, or both is rounded. In some embodiments, the primary second portion 120A and the secondary second portion 120B are generally symmetric about the first portion 110. In some embodiments, the primary second portion 120A and the secondary second portion 120B are asymmetric about first portion 110. In some embodiments, the primary second portion 120A and the secondary second portion 120B are generally coplanar. In some embodiments, the primary second portion 120A and the secondary second portion 120B are generally perpendicular. In some embodiments, an angle between the primary second portion 120A and the secondary second portion 120B at the intersection of the first portion 110 and the second portion 120 is about 90 degrees to about 180 degrees.

In some embodiments, the primary second portion 120A is configured to be inserted beneath an upper eyelid 140A of the patient and wherein the secondary second portion 120B is configured to be inserted beneath a lower eyelid 140B of the patient. Alternatively, in some embodiments, the primary second portion 120A is configured to be inserted beneath a lower eyelid 140B of the patient and wherein the secondary second portion 120B is configured to be inserted beneath an upper eyelid 140A of the patient.

### Medicament Surface

As shown in FIGS. 1-8, 21, 22, 23, 24A-24B, and 25A-25B the first portion 110 comprises a medicament surface 130. In some embodiments, the medicament surface 130 is configured to receive a medicament. In some embodiments, the medicament is solid, liquid, or a paste. Further per FIGS. 1-8, the medicament surface 130 is at an intersection of the first portion 110 and the second portion 120. Additionally, per FIGS. 19A-B, in some embodiments, the medicament surface 130 is offset from the first portion 110. In some embodiments, the medicament surface 130 is offset from the first portion 110 by a distance of about 1 mm, 2 mm, 3 mm, 4 mm, 5 mm, 6 mm, 7 mm, 8 mm, 9 mm, 10 mm, or more including increments therein. In some embodiments, the medicament surface 130 is offset from the first portion 110, whereas a gap exists between the first portion 110 and the medicament surface 130. In some embodiments, the medicament surface 130 extends from the intersection of the first portion 110 and the second portion 120 along the first portion 110. In some embodiments, the medicament surface 130 extends from the intersection of the first portion 110 and the second portion 120 along the first portion 110 by 1 mm, 2 mm, 3 mm, 4 mm, 5 mm, 6 mm, 7 mm, 8 mm, 9 mm, 10 mm, or more including increments therein. In some embodiments, the medicament surface 130 has a length of about 1 mm, 2 mm, 3 mm, 4 mm, 5 mm, 6 mm, 7 mm, 8 mm, 9 mm, 10 mm, or more including increments therein. In some embodiments, the medicament surface 130 contacts at least a section of the eyelid 140 of the patient. In some embodiments, the medicament surface 130 contacts at least a section of the eyelid 140 of the patient when the second portion 120 is inserted beneath the eyelid 140 of the patient. In some embodiments, the section of the eyelid 140 of the patient is the lid margin of the eyelid 140 of the patient.

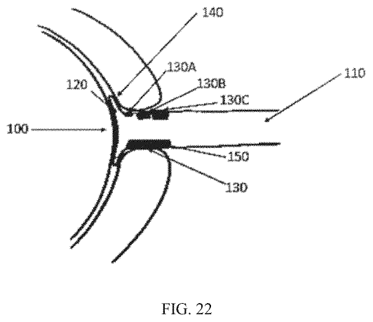

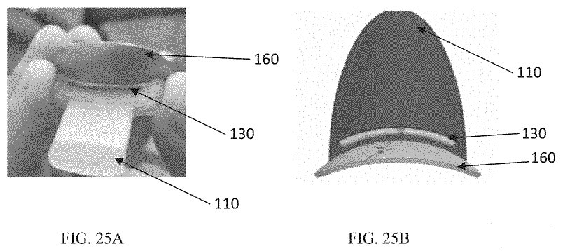

In some embodiments, the medicament surface 130 is placed between the eyelids and a sealing element 120 can prevent contact with the ocular surface. In some embodiments, the medicament surface 130 is on the distal end of the first portion 110 as can been seen in FIG. 22. The medicament surface 130 can be placed at various distances from the plane of the cornea such that it is targeting diseases at different areas on the lid margin. For example, for treating lid wiper epitheliopathy (LWE) which is a disease of the posterior part of the lid margin, requiring the medicament surface 130A to be located at the most distal part of the first portion 110. Meibomian gland disorder is a diseased of the middle of the lid margin, can be treated by placing the medicament surface 130B more anterior on the first portion 110. Demodex which affects the base of the eyelashes can be treated by placing the medicament surface 130C more anterior on the distal part of the first portion 110 as is shown in FIG. 22. The three positions, anterior, middle and distal of the medicament surface can be seen in FIG. 22. For example, having the medicament surface 130 placed 500 microns from the distal end of the first portion 110 (FIG. 25B) places the medicament at the root of the eyelashes (e.g., where Demodex can affect the lid margin). FIG. 25A demonstrates where the medicament is contained in the medicament recession, thereby avoiding spreading the medicament to more distal eyelid regions (e.g., where the meibomian glands are located). As can be seen in FIG. 25A and FIG. 25B, the second portion can comprise a shield 160 to prevent the medicament from contacting the ocular surface.

In some embodiments, the medicament surface 130 can be placed in a medicament recession along the first portion 110. In some embodiments, the medicament recession has a distance from the distal end, a depth, width and length specific to the targeted region of the lid margin. The medicament recession can be used to control the amount of medicament 150 provided to the lid margin, control the placement of the medicament 150 on the lid margin, or assist in preventing leakage beyond the recession.

In some embodiments, the medicament surface 130 is concave. In some embodiments, the medicament surface 130 is flat. In some embodiments, the medicament surface 130 is cylindrical. In some embodiments, the medicament surface 130 is spherical. In some embodiments, the medicament surface 130 comprises a medicament 150 embedded within the medicament surface 130. In some embodiments, the medicament 150 within the medicament surface 130 is released from the medicament surface 130 over time. In some embodiments, the medicament 150 within the medicament surface 130 is released from the medicament surface 130 over a period of time of about 0.1 minutes to about 60 minutes. In some embodiments, the medicament surface 130 comprises a medicament material having the medicament 150 and adhered to the first portion 110. In some embodiments, the medicament material releases the medicament 150 when wetted, exposed to air, or both. In some embodiments, the medicament surface 130 is offset from the first portion 110.

In some embodiments, the medicament surface 130 is demarcated on the first portion 110. In some embodiments, the medicament surface 130 is demarcated by a label, a boundary, a surface texture, or any combination thereof. In some embodiments, at least a portion of the medicament surface 130 is surrounded by a protrusion, a recess, or both. In some embodiments, at least a portion of the medicament surface 130 comprises a protrusion, a recess, or both. In some embodiments, at least a portion of the medicament surface 130 has a surface texture having a hydrophobicity of greater than or less than the first portion 110 outside the medicament surface 130. In some embodiments, at least a portion of the medicament surface 130 has porosity to absorb the medicament 150. In some embodiments, at least a portion of the medicament surface 130 has porosity to release the medicament 150 under compression. In some embodiments, at least a portion of the medicament surface 130 is coated or covered by a material having a hydrophobicity of greater than or less than the first portion 110 outside the medicament surface 130. In some embodiments, the medicament surface 130 is not demarcated on the first portion 110.

In some embodiments, the medicament surface 130 comprises a primary medicament surface 130A at an intersection of the first portion 110 and the primary second portion 120A and a secondary medicament surface 130B at an intersection of the first portion 110 and the secondary second portion 120B. In some embodiments, the primary medicament surface 130A contacts at least a portion of an upper eyelid 140A of the patient when the second portion 120 is inserted beneath the eyelid 140 of the patient. In some embodiments, the secondary medicament surface 130B contacts at least a portion of a lower eyelid 140B of the patient when the second portion 120 is inserted beneath the eyelid 140 of the patient. In some embodiments, the medicament surface 130 is coplanar with at least one of the first portion 110 and the second portion 120. In some embodiments, at least a portion of the medicament surface 130 is perpendicular to at least one of the first portion 110 and the second portion 120.

As can be seen in FIG. 22, in some embodiments, one or more medicament recessions can use used to specifically target a location on the lid margin. In some embodiments, the medicament recessions are placed to line up with a specific location on the lid margin when the device is placed. In some embodiments, the medicament recessions are located at an anterior, middle or distal position along the first portion 110. For example, for treating lid wiper epitheliopathy (LWE) which is a disease of the posterior part of the lid margin, requiring the medicament recession and medicament surface 130C to be located at the most distal part of the first portion 110. Meibomian gland disorder is a diseased of the middle of the lid margin, can be treated by placing the medicament recession and medicament surface 130B more anterior on the first portion 110. Demodex which affects the base of the eyelashes can be treated by placing the medicament recession and medicament surface 130A more anterior on the distal part of the first portion 110 as is shown in FIG. 22.

In some embodiments, the medicament recession is used in conjunction with a medicament that is not compatible with the ocular surface. For example, the medicament recession can be used to apply keratolytic agents that require physical separation from the ocular surface. In some embodiments, the device physically separates the eyelid from the ocular surface and allows for the treatment of the eyelid separate from the ocular surface. The medicament can comprise salicylic acid or urea at high concentrations. For example, the medicament can comprise salicylic acid or urea at high concentrations for the treatment of meibomian gland disorder and lid wiper epitheliopathy. The medicament can comprise tea tree oil at high concentrations (e.g. 10 weight % to 50 weight %). The medicament can comprise tea tree oil at high concentrations to treat demodex such as 10 weight % to 50 weight %. The medicament can comprise therapeutic agents that are not tolerable on an ocular surface at a high concentration.

In some embodiments, at least one of the medicament recessions are positioned on the first portion 110 within 0 micrones, 100 micrones, 200 micrones, 300 micrones, 400 micrones, 500 micrones, 600 micrones, 700 micrones. 800 micrones. 900 micrones 1 mm, 2 mm, 3 mm, 4 mm, 5 mm, 6 mm, 7 mm, 8 mm, 9 mm, 10 mm, or more including increments therein from the intersection of the first portion 110 and the second portion 120. In some embodiments, at least one of the medicament recessions has a length of about 0 micrones, 100 micrones, 200 micrones, 300 micrones, 400 micrones, 500 micrones, 600 micrones, 700 micrones. 800 micrones. 900 micrones 1 mm, 2 mm, 3 mm, 4 mm, 5 mm, 6 mm, 7 mm, 8 mm, 9 mm, 10 mm, 15 mm, 20 mm, or more including increments therein. In some embodiments, a ratio between a thickness of the second portion 120 and a height of the medicament recession is about 10:1 to about 1.5:1. In some embodiments, a ratio between a thickness of the first portion 110 and a depth of the medicament recession is about 10:1 to about 1.5:1. Width of recess can be 200 micrones and 6000 micrones, depth can be between 1 micrones and 2000 micrones, length can be between 0.5 mm-3 mm. The recess can ideally be curved to follow the shape of the lid. The recess can also be through and through connecting with the recess at the other side of the 110. Or two recess can be at different distance or dimensions for upper and lower lid based on their relative anatomy.

In some embodiments, the widths, lengths, heights, depths, and offsets described herein are measured as a minimum depth, a maximum depth, or an average depth.

### Sealing Protrusions and Recesses

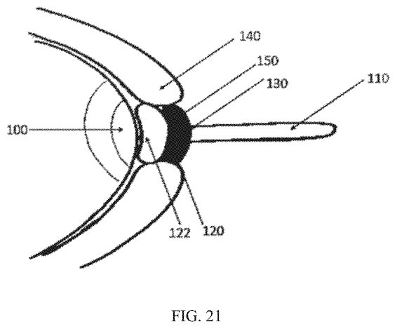

As shown in FIG. 21, the device can comprise a first portion 110 comprising a handle and a second portion 120 comprising a sealing element 122 in contact with the ocular surface 100. In some embodiments, the sealing element 122 is in contact with the posterior portion of the eyelid margin 140. In some embodiments, the device comprises a handle and a sealing element. In some embodiments, the medicament 150 can be placed at a distal end of the handle. In some embodiments, the medicament 150 can be placed adjacent to the sealing element 122. In some embodiments, the medicament surface 130 is in contact with the middle or anterior portion of the eyelid margin 140. The medicament 150 can be applied to one or more target locations on an eyelid 140 by placing the device at different depths over the eyelid margin (e.g. the eyelashes at the anterior portion of the eyelid margin or the meibomian glands at the posterior portion of the eyelid margin as can be seen in FIG. 26.) The medicament 150 can be applied at the anterior portion of the eyelid margin by placing medicament surface 130 further from the sealing element 122 such that the medicament is placed on the eyelashes. The medicament 150 can be applied at the anterior portion of the eyelid margin by placing the medicament surface 130 closer to the sealing element 122 such that the medicament is placed on one or more meibomian gland orifice. For example, having the medicament surface 130 placed 500 microns from the distal end of the handle of the first portion 110 places the medicament at the root of the eyelashes (e.g., where Demodex can affect the lid margin).

The sealing element can comprise a part of the distal end of the first portion 110. The sealing element can be located at various regions of the lid margin. In some embodiments, the medicament 150 is anterior to the sealing element to prevent the medicament 150 from reaching the ocular surface 100. For example, if the medicament 150 is applied to treat Demodex on the eyelashes of the lid margin, the sealing element can be located at the middle of the lid margin. If the medicament 150 is applied to treat meibomian gland disorder, the sealing element can be located at the posterior portion of the lid margin. The sealing element can be a sponge. The sealing element can have adhesive properties. The sealing element be used in conjunction with mechanical pressure.

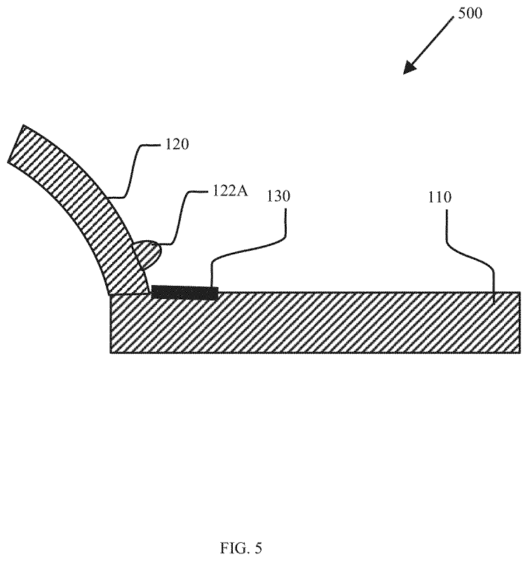

As shown in FIGS. 5-6, 15, 19A-B, 21, and 22 the second portion 120 comprises a sealing protrusion 122A 122B 122C. In some embodiments, the sealing protrusion 122A 122B 122C is configured to prevent a medicament 150 deposited on the medicament surface 130 from contacting the eye of the patient. In some embodiments, the sealing protrusion 122A 122B 122C is configured to prevent a medicament 150 deposited on the medicament surface 130 from contacting the eye of the patient by acting as a barrier for medicament 150 traveling towards the eye of the patient. In some embodiments, the sealing protrusion 122A 122B 122C is configured to prevent a medicament 150 deposited on the medicament surface 130 from contacting the eye of the patient by absorbing medicament 150 traveling towards the eye of the patient.

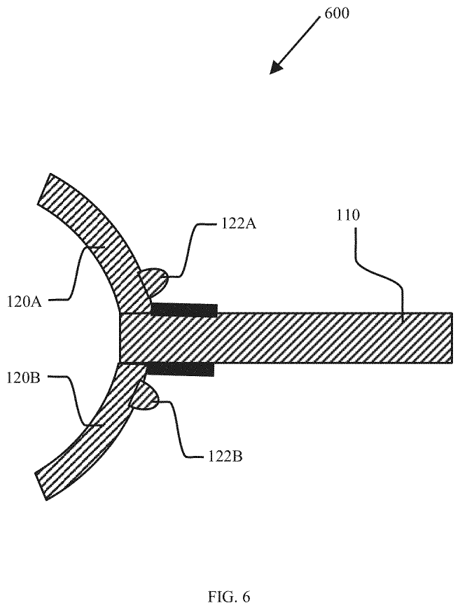

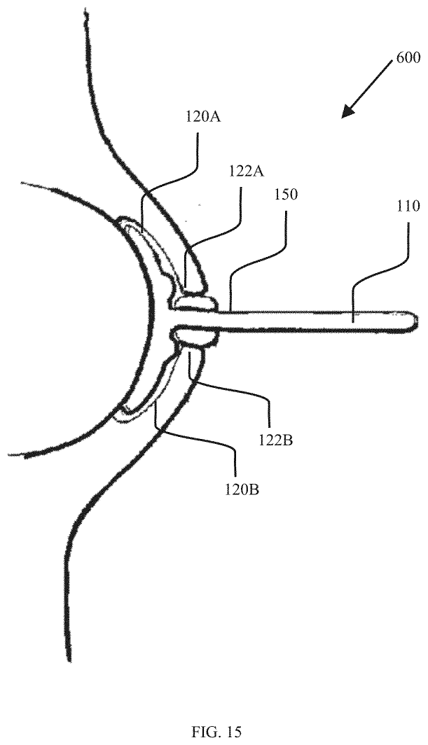

As shown in FIG. 5 the second portion 120 comprises a single primary sealing protrusion 122A. As shown in FIGS. 6 and 15 the primary second portion 120A comprises a primary sealing protrusion 122A and the secondary second portion 120B comprises a secondary sealing protrusion 122B. Per FIGS. 19A-B, the second portion 120 comprises a tertiary sealing protrusion 122C. In some embodiments, at least one of the primary sealing protrusion 122A and the secondary second portion 120B extend parallel to an upper surface of the first portion 110. In some embodiments, the tertiary sealing protrusion 122C extends perpendicularly to the upper surface of the first portion 110. In some embodiments, the second portion comprises one or more of the primary, secondary, and tertiary protrusions 122A 122B 122C. In some embodiments, the second portion does not comprise at least one of the primary, secondary, and tertiary protrusions 122A 122B 122C. In some embodiments, two or more of the primary, secondary, and tertiary protrusions 122A 122B 122C are interconnected.

Alternatively, in some embodiments, the second portion 120 comprises a plurality of sealing protrusions 122A 122B 122C. Alternatively, the primary second portion 120A comprises a plurality of primary sealing protrusions 122A and the secondary second portion 120B comprises a plurality of secondary sealing protrusions 122B. In some embodiments, the primary second portion 120A comprises the primary sealing protrusion 122A, wherein the secondary second portion 120B does not comprise the secondary sealing protrusion 122B. In some embodiments, the primary second portion 120A does not comprise the primary sealing protrusion 122A, wherein the secondary second portion 120B comprises the secondary sealing protrusion 122B.

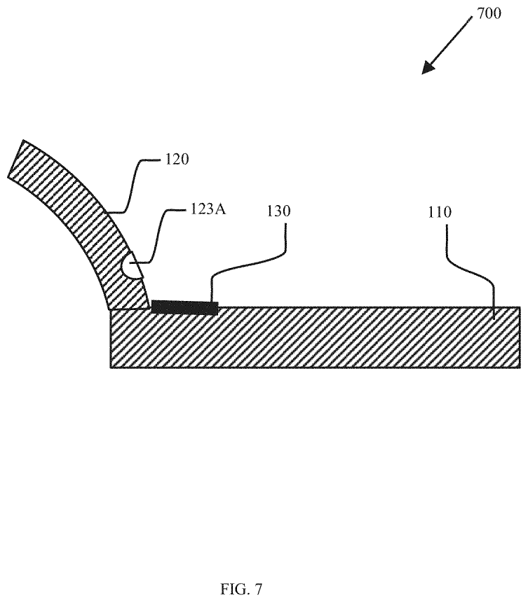

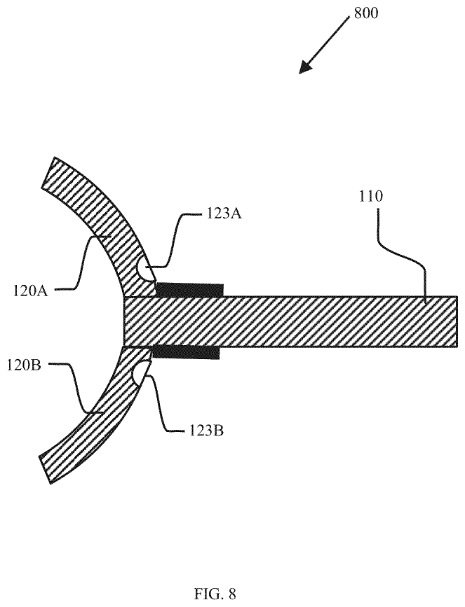

As shown in FIGS. 7-8 the second portion 120 comprises a sealing recess 123A 123B. In some embodiments, the sealing recess 123A 123B is configured to prevent a medicament 150 deposited on the medicament surface 130 from contacting the eye of the patient. In some embodiments, the sealing recess 123A 123B is configured to prevent a medicament 150 deposited on the medicament surface 130 from contacting the eye of the patient by acting as a barrier for any medicament 150 traveling towards the eye of the patient. As shown in FIG. 7 the second portion 120 comprises a single sealing recess 123A 123B. Alternatively, in some embodiments, the second portion 120 comprises a plurality of the sealing recesses 123A 123B. As shown in FIG. 8 the primary second portion 120A comprises a primary sealing recess 123A and the secondary second portion 120B comprises a secondary sealing recess 123B. Alternatively, the primary second portion 120A comprises a plurality of primary sealing recesses 123A and the secondary second portion 120B comprises a plurality of secondary sealing recesses 123B. In some embodiments, the primary second portion 120A comprises a primary sealing recess 123A, wherein the secondary second portion 120B does not comprise the secondary sealing recess 123B. In some embodiments, the primary second portion 120A does not comprise the primary sealing recess 123A, wherein the secondary second portion 120B comprises the secondary sealing recess 123B. In some embodiments, at least one of the primary sealing recess 123A and the secondary second portion 120B extend parallel to an upper surface of the first portion 110. In some embodiments, the tertiary sealing recess 123C extends perpendicular to the upper surface of the first portion 110. In some embodiments, the second portion comprises one or more of the primary, secondary, and tertiary recess 123A 123B 123C. In some embodiments, the second portion does not comprise at least one of the primary, secondary, and tertiary recess 123A 123B 123C. In some embodiments, two or more of the primary, secondary, and tertiary recess 123A 123B 123C are interconnected.

In some embodiments, at least one of the primary and secondary sealing protrusions 122A 122B is positioned on the second portion 120 within 1 mm, 2 mm, 3 mm, 4 mm, 5 mm, 6 mm, 7 mm, 8 mm, 9 mm, 10 mm, or more including increments therein from the intersection of the first portion 110 and the second portion 120. In some embodiments, at least one of the primary and secondary sealing protrusions 122A 122B has a length of about 1 mm, 2 mm, 3 mm, 4 mm, 5 mm, 6 mm, 7 mm, 8 mm, 9 mm, 10 mm, 15 mm, 20 mm, or more including increments therein. In some embodiments, the tertiary sealing protrusions 122C is positioned distally from the medicament surface 130 by about 1 mm, 2 mm, 3 mm, 4 mm, 5 mm, 6 mm, 7 mm, 8 mm, 9 mm, 10 mm, or more including increments therein. In some embodiments, the tertiary sealing protrusions 122C has a width of about 1 mm, 2 mm, 3 mm, 4 mm, 5 mm, 6 mm, 7 mm, 8 mm, 9 mm, 10 mm, 15 mm, 20 mm, or more including increments therein.

In some embodiments, at least one of the primary and secondary sealing recess 123A 123B is positioned on the second portion 120 within 1 mm, 2 mm, 3 mm, 4 mm, 5 mm, 6 mm, 7 mm, 8 mm, 9 mm, 10 mm, or more including increments therein from the intersection of the first portion 110 and the second portion 120. In some embodiments, at least one of the primary and secondary sealing recess 123A 123B has a length of about 1 mm, 2 mm, 3 mm, 4 mm, 5 mm, 6 mm, 7 mm, 8 mm, 9 mm, 10 mm, 15 mm, 20 mm, or more including increments therein. In some embodiments, a ratio between a thickness of the second portion 120 and a height of the sealing protrusion 122A 122B 122C is about 10:1 to about 1.5:1. In some embodiments, a ratio between a thickness of the second portion 120 and a depth of the sealing recess 123A 123B 123C is about 10:1 to about 1.5:1.

In some embodiments, the widths, lengths, heights, depths, and offsets described herein are measured as a minimum depth, a maximum depth, or an average depth.

### Absorbing Materials

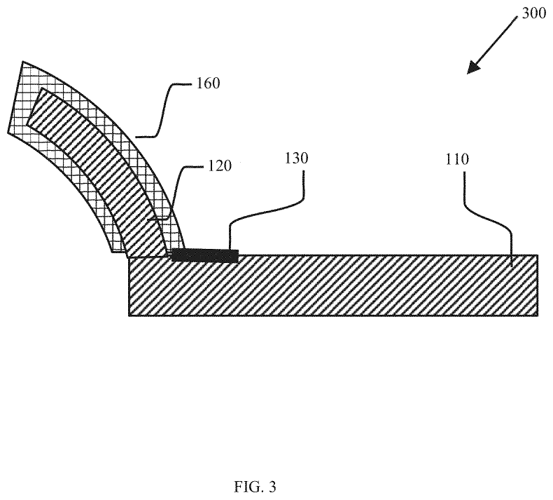

As shown in FIGS. 3-4 and 9-14 the second portion 120 comprises a coating of an absorbing material 160. In some embodiments, the absorbing material 160 is configured to absorb the medicament 150. In some embodiments, the absorbing material 160 is configured to absorb a sufficient quantity of the medicament 150, such that at least a portion of the volume of the medicament 150 disposed on the medicament surface 130 is absorbed by the absorbing material 160. In some embodiments, the absorbing material 160 is configured to absorb a sufficient quantity of the medicament 150, such that at least a majority of the volume of the medicament 150 disposed on the medicament surface 130 is absorbed by the absorbing material 160. In some embodiments, the coating of the absorbing material 160 is configured to prevent a medicament 150 deposited on the medicament surface 130 from contacting the eye of the patient. In some embodiments, the coating of the absorbing material 160 is configured to prevent a medicament 150 deposited on the medicament surface 130 from contacting the eye of the patient by acting as a barrier for any medicament 150 traveling towards the eye of the patient. In some embodiments, the absorbing material 160 is sufficiently porous to absorb the medicament 150. In some embodiments, the absorbing material 160 comprises a metal, a plastic, a ceramic, a fiber, or any combination thereof.

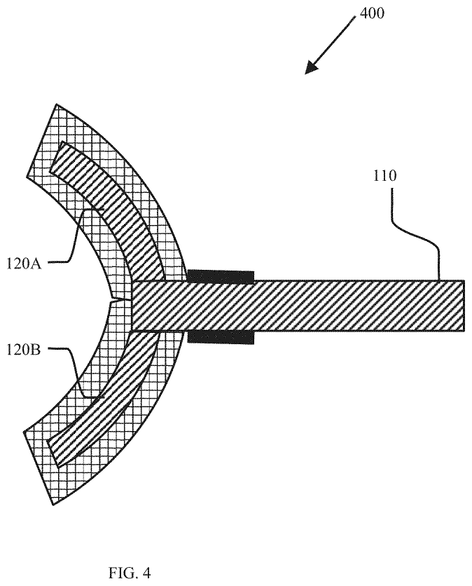

As shown in FIG. 3 the second portion 120 comprises a single coating of the absorbing material 160. As shown in FIG. 4 the primary second portion 120A and the secondary second portion 120B comprise the coating of the absorbing material 160. In some embodiments, at most a section of the second portion 120 is coated by the absorbing material 160. In some embodiments, at most a section of the primary second portion 120A, the secondary second portion 120B, or both is coated by the absorbing material 160. In some embodiments, the primary second portion 120A comprises the coating of the absorbing material 160, wherein the secondary second portion 120B does not comprise the coating of the absorbing material 160. In some embodiments, the primary second portion 120A does not comprise the coating of the absorbing material 160, wherein the secondary second portion 120B comprises the coating of the absorbing material 160. In some embodiments, the coating of the absorbing material 160 is positioned on the second portion 120 within 1 mm, 2 mm, 3 mm, 4 mm, 5 mm, 6 mm, 7 mm, 8 mm, 9 mm, 10 mm, or more including increments therein from the intersection of the first portion 110 and the second portion 120. In some embodiments, a ratio between a thickness of the second portion 120 and a thickness of the coating of the absorbing material 160 is about 10:1 to about 1.5:1.

In some embodiments, the distance of the coating of the absorbing material 160 from the first portion 110 is measured as a minimum distance, a maximum distance, or an average distance. In some embodiments, the thickness of the second portion 120 is measured as a minimum thickness, a maximum thickness, or an average thickness. In some embodiments, the thickness of the coating of the absorbing material 160 is measured as a minimum thickness, a maximum thickness, or an average thickness.

### Methods for Applying a Medicament to an Eyelid

Another aspect provided herein is a method for applying a medicament to an eyelid of a patient comprising applying a medicament to a medicament surface, inserting the second portion beneath the eyelid of the patient, and removing the second portion from beneath the eyelid of the patient. In some embodiments, the medicament surface is on a first portion and at an intersection of the first portion and a second portion of an eyelid medicament delivery device. In some embodiments, inserting the second portion beneath the eyelid of the patient is performed such that the medicament on the medicament surface contacts at least a section of the eyelid of the patient.

In some embodiments, at least part of the second portion is covered by an absorbing material. In some embodiments, the method further comprises absorbing the medicament by the absorbing material. In some embodiments, the method further comprises preventing the medicament from contacting the eye of the patient by the absorbing material.

In some embodiments, the second portion comprises a primary second portion and a secondary second portion. In some embodiments, inserting the second portion beneath the eyelid of the patient comprises inserting a primary second portion beneath an upper eyelid of the patient and inserting a secondary second portion beneath a lower eyelid of the patient. In some embodiments, inserting the second portion beneath the eyelid of the patient comprises inserting the second portion beneath an upper eyelid of the patient inserting the second portion beneath a lower eyelid of the patient, or both. In some embodiments, inserting the second portion beneath the eyelid of the patient is performed using only one hand. In some embodiments, the medicament surface comprises a primary medicaments surface and a secondary medicament surface. In some embodiments, applying the medicament to the medicament surface comprises applying the medicament to the primary medicament surface, the secondary medicament surface, or both. In some embodiments, applying the medicament to the medicament surface comprises applying the medicament to the primary medicament surface and not applying the medicament to the secondary medicament surface. In some embodiments, applying the medicament to the medicament surface comprises applying the medicament to the secondary medicament surface and not applying the medicament to the primary medicament surface.

In some embodiments, the method further comprises maintaining the second portion beneath the eyelid for a period of time such that the medicament on the medicament surface contacts at least the section of the eyelid of the patient for the period of time. In some embodiments, the period of time is about 0.5 minutes, 1 minute, 1.5 minutes, 2 minutes, 2.5 minutes, 3 minutes, 4 minutes, 5 minutes, 10 minutes, 15 minutes, 20 minutes, 25 minutes, 30 minutes, or more including increments therein. In some embodiments, maintaining the second portion beneath the eyelid for the period of time such that the medicament on the medicament surface contacts at least the section of the eyelid of the patient enables the medicament to be absorbed by the eyelid of the patient. In some embodiments, maintaining the second portion beneath the eyelid for the period of time occurs without physical intervention or contact by a caregiver.

### Kit for Applying a Medicament to an Eyelid

Another aspect provided herein is a kit for applying a medicament to an eyelid comprising: the device or apparatus herein, and a medicament. In some embodiments, the kit further comprises an instruction sheet, a container, a q-tip, an anesthetic drop, a washing solution, a lid wipe, or any combination thereof. In some embodiments, the kit comprises a plurality of instruction sheets, containers, q-tips, anesthetic drops, washing solutions, lid wipes, or any combination thereof.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure.

1-212. (canceled)

213. A method of administering a medicament to an eyelid of an individual, the method comprising:
(a) providing a device comprising:
(i) a first portion having a distal end and a proximal end;
(ii) a second portion configured to be inserted beneath an eyelid of the individual;
(iii) a medicament surface with a medicament configured thereon, the medicament surface being on the first portion and at an intersection of the first portion and the second portion; and
(b) providing the medicament surface in proximity to at least a section of the eyelid of the individual, thereby providing the medicament to the eyelid.

214. The method of claim 213, wherein the second portion is inserted beneath the eyelid of the individual, and wherein when the second portion is inserted beneath the eyelid of the individual the medicament surface is positioned in proximity of the eyelid of the individual.

215. The method of claim 213, further comprising applying the medicament to the medicament surface.

216. The method of claim 213, wherein the individual provides the medicament surface in proximity to the eyelid.

217. The method of claim 213, further comprising inserting the second portion beneath the eyelid of the individual such that the medicament on the medicament surface contacts at least a section of the eyelid of the individual.

218. The method of claim 217, further comprising inserting the second portion beneath an upper eyelid of the individual, inserting the second portion beneath a lower eyelid of the individual, or both.

219. The method of claim 218, further comprising removing the second portion from beneath the eyelid of the individual.

220. The method of claim 213, wherein at least one of the first portion and the second portion are formed of a material that is impermeable to the medicament.

221. The method of claim 213, wherein only the medicament surface is formed of a material that is permeable to the medicament.

222. The method of claim 213, further comprising preventing the medicament from contacting the eye of the individual with an absorbing material.

223. The method of claim 213, wherein the second portion is further configured to prevent the medicament deposited on the medicament surface from contacting the eye of the individual.

224. The method of claim 213, wherein the second portion further comprises a sealing protrusion, a sealing recess, or both, and wherein at least one of the sealing protrusion and the sealing recess prevents the medicament from contacting the eye of the individual.

225. The method of claim 213, wherein at least part of the second portion is covered by an absorbing material, the absorbing material being configured to absorb the medicament.

226. The method of claim 225, wherein the absorbing material is configured to prevent the medicament deposited on the medicament surface from contacting the eye of the individual.

227. The method of claim 213, wherein the second portion comprises a sealing protrusion, a sealing recess, or both.

228. The method of claim 227, wherein at least one of the sealing protrusion and the sealing recess is configured to prevent the medicament deposited on the medicament surface from contacting the eye of the individual.

229. The method of claim 213, wherein at least one of the first portion and the second portion are formed of a biocompatible material.

230. The method of claim 229, wherein the biocompatible material comprises nylon, silicone, hydrogel, silicone-hydrogel, shell, Glass, PMMA, Acrylic, Metal polycarbonate, polyester, polyethylene, polyethylene terephthalate, polypropylene, polyurethane, polyvinyl chloride, polyvinylidene chloride, or any combination thereof.

231. The method of claim 213, wherein the first portion is shaped and dimensioned to allow close contact between an eye lid margin of the individual and the medicament surface for an extended period of time.

232. The method of claim 231, wherein the extended period of time is about 0.25 minute to about 60 minutes.
